(19) United States
(12) Patent Application Publication
(10) Pub. No.: US 2023/0047809 A1
(43) Pub. Date: Feb. 16, 2023

(54) PULMONARY DELIVERY OF ANTIBODIES

(71) Applicants: SANOFI, Paris (FR); UNIVERSITE DE TOURS, Tours Cedex 1 (FR); INSTITUT NATIONAL DE LA SANTÉ ET DE LA RECHERCHE MÉDICALE (INSERM), Paris (FR)

(72) Inventors: Sylvain HUILLE, Paris (FR); Heloise AUDAT, Paris (FR); Alexie MAYOR, Beaugency (FR); Nathalie HEUZÉ-VOURC'H, Tours (FR); Renaud RESPAUD, Tours (FR)

(21) Appl. No.: 17/796,568

(22) PCT Filed: Feb. 1, 2021
(86) PCT No.: PCT/EP2021/052315

§ 371 Date: Jul. 29, 2022

(30) Foreign Application Priority Data
Jan. 31, 2020 (EP) 20305091.9

(51) Int. Cl. A61K 9/00; A61K 47/18; A61K 47/68; A61K 47/10

(52) U.S. Cl. CPC A61K 9/0078; A61K 47/183; A61K 47/68; A61K 47/10

(57) ABSTRACT

The present invention relates to the pulmonary delivery of antibodies or antibody derivatives.

### TECHNICAL FIELD OF THE INVENTION

The present invention relates to the pulmonary delivery of antibodies or antibody derivatives.

### BACKGROUND OF THE INVENTION

Attention to therapeutic antibodies has been dramatically increasing year by year, as their highly specific targeting of antigens can provide very effective treatment of various diseases.

Pulmonary delivery of antibodies, such as monoclonal antibodies, could represent an attractive, non-invasive alternative to parental delivery. The pulmonary route of administration has proven to be effective in local and systemic delivery of miscellaneous drugs and biopharmaceuticals to treat pulmonary and non-pulmonary diseases.

However, administration of proteins, such as antibodies, to the lungs is associated with many challenges, such as the need for an appropriate formulation of the antibodies to overcome strong intermolecular/inter-particle interactions and physico-chemical degradation leading, for example, to aggregation and potentially to loss of biological/therapeutic activity and/or safety issues. For example, proteins can be sensitive towards aerosolization-associated shear stress and/or increase in temperature and/or may exhibit decreased stability at the air-liquid interface in an aerosol.

Accordingly, it was an object of the present invention to identify formulation systems that are suitable for pulmonary delivery of antibodies and that help to maintain stability and activity of the antibodies upon aerosolization/nebulization.

### SUMMARY OF THE INVENTION

In one aspect, the present invention relates to an aerosol comprising droplets comprising a liquid formulation, wherein the liquid formulation comprises
(i) an antibody or antibody derivative,
(ii) a buffering agent selected from the group consisting of acetate, histidine and combinations thereof, and
(iii) an aqueous medium; and

wherein the liquid formulation has a pH which is equal to or lower than about 5.5.

In one embodiment, the antibody or antibody derivative is an IgG1 antibody or antibody derivative.

In one embodiment, the antibody is a monoclonal antibody.

In one embodiment, the liquid formulation does not comprise citrate.

In one embodiment, the liquid formulation has a pH in the range of from about 3.5 to about 5.5.

In one embodiment, the buffering agent is acetate, and the liquid formulation has a pH which is lower than about 5.0 or lower than about 4.5. In one embodiment, the buffering agent is acetate, and the liquid formulation has a pH which is in the range of between about 3.5 and lower than about 4.5 or of between about 3.7 and about 4.3 or of between about 3.8 and about 4.2 or of between about 3.9 and about 4.1. In one embodiment, the liquid formulation has a pH of about 4.0.

In one embodiment, the liquid formulation further comprises a surfactant. In one embodiment, the surfactant is selected from the group consisting of polysorbates, poloxamers, polyoxyethylenealkylethers, alkylphenolpolyoxyethylene and sodium dodecyl sulfate.

In one embodiment, the concentration of the surfactant in the liquid formulation is equal to or lower than about 0.1% (w/v) or equal to or lower than about 0.05% (w/v).

In one embodiment, the liquid formulation does not comprise any surfactant.

In one embodiment, the liquid formulation does not comprise NaCl.

In one embodiment, the liquid formulation does not comprise any non-buffering salt.

In one embodiment, the droplets have an average diameter in the range of from about 0.5 μm to about 5 m, from about 0.5 μm to about 4.5 m, from about 0.5 μm to about 4 m, from about 0.5 m to about 3.5 μm or from about 0.5 μm to about 3 μm.

In one embodiment the droplets have an average diameter below 5 μm. In one embodiment the droplets have an average diameter below 4.5 μm. In one embodiment the droplets have an average diameter below 4.0 μm. In one embodiment the droplets have an average diameter below 3.5 μm. In one embodiment the droplets have an average diameter below 3.0 μm.

In another aspect, the present invention relates to a method of preparing an aerosol comprising droplets comprising a liquid formulation, said method comprising the steps:
(i) providing a liquid formulation as defined above,
(ii) nebulizing the liquid formulation provided in step (i) by means of a nebulizer, thereby preparing the aerosol.

In one embodiment, the nebulizer is a mesh nebulizer.

In one embodiment, the method further comprises, between steps (i) and (ii), the steps of:
(ia) lyophilizing the liquid formulation provided in step (i), thereby providing a lyophilized powder, and
(ib) reconstituting the liquid formulation provided in step (i) by adding an appropriate amount of an aqueous medium to the lyophilized powder provided in step (ia).

In another aspect, the present invention relates to an aerosol comprising droplets comprising a liquid formulation, wherein the aerosol is obtainable by the method as defined above. In one embodiment, the droplets have an average diameter in the range of from about 0.5 μm to about 5 μm or from about 0.5 μm to about 3 μm.

In another aspect, the present invention relates to the liquid formulation as defined above or the aerosol as defined above for use in a method of delivering an antibody or antibody derivative to the lungs of a subject, wherein the aerosol is administered to the subject by inhalation or the liquid formulation is administered to the subject by inhalation via a nebulizer.

In one embodiment, the antibody or antibody derivative is an IgG1 antibody or antibody derivative.

In one embodiment, the nebulizer is a mesh nebulizer.

In another aspect, the present invention relates to the liquid formulation as defined above for use in a method of treating or preventing a disease in a subject, wherein the aerosol is administered to the subject by inhalation or the liquid formulation is administered to the subject by inhalation via a nebulizer.

In one embodiment, the disease is a lung disease.

In one embodiment, the nebulizer is a mesh nebulizer.

In another aspect, the present invention relates to a method of delivering an antibody or antibody derivative to the lungs of a subject, said method comprising administering to the subject an effective amount of the aerosol as defined above by inhalation or administering to the subject an effective amount of the liquid formulation as defined above by inhalation via a nebulizer.

In one embodiment, the antibody or antibody derivative is an IgG1 antibody or antibody derivative.

In one embodiment, the nebulizer is a mesh nebulizer.

In another aspect, the present invention relates to a method of treating or preventing a disease in a subject, said method comprising administering to the subject an effective amount of the aerosol as defined above by inhalation or administering to the subject an effective amount of the liquid formulation as defined above by inhalation via a nebulizer.

In one embodiment, the disease is a lung disease.

In one embodiment, the nebulizer is a mesh nebulizer.

In one embodiment, the lung disease referred to above is selected from the group consisting of asthma, chronic obstructive pulmonary disease (COPD), lung cancer, cystic fibrosis (CF), interstitial lung disease (ILD), such as idiopathic pulmonary fibrosis, hypersensitivity pneumonitis, sarcoidosis and asbestosis, bacterial infections, viral infections, acute respiratory distress syndrome (ARDS), pulmonary alveolar proteinosis (PAP), acute bronchitis, bronchiolitis obliterans and pulmonary hypertension.

In another aspect, the present invention relates to a nebulizer comprising a liquid formulation as defined above.

In one embodiment, the nebulizer is a mesh nebulizer.

In another aspect, the present invention relates to a kit comprising
(i) a container comprising the liquid formulation as defined above or a powder obtainable by lyophilization of the liquid formulation, and
(ii) a nebulizer.

In one embodiment, the nebulizer is a mesh nebulizer.

In another aspect, the present invention relates to the use of a liquid formulation as defined above for preparing an aerosol by nebulization by means of a nebulizer.

In one embodiment, the nebulizer is a mesh nebulizer.

In another aspect, the present invention relates to the use of a buffering agent selected from the group consisting of acetate, histidine and combinations thereof for increasing the stability of an antibody or antibody derivative upon nebulization of a liquid formulation comprising the antibody or antibody derivative by means of a nebulizer, wherein the buffering agent is included in the liquid formulation prior to nebulization.

In one embodiment, the antibody or antibody derivative is an IgG1 antibody or antibody derivative.

In one embodiment, the nebulizer is a mesh nebulizer.

In one embodiment, the liquid formulation has a pH which is equal to or lower than about 5.5.

In one embodiment, the antibody is a monoclonal antibody.

In one embodiment, the liquid formulation does not comprise citrate.

In one embodiment, the liquid formulation has a pH in the range of from about 3.5 to about 5.5.

In one embodiment, the buffering agent is acetate, and the liquid formulation has a pH which is lower than about 5.0 or lower than about 4.5. In one embodiment, the buffering agent is acetate, and the liquid formulation has a pH which is in the range of between about 3.5 and lower than about 4.5 or of between about 3.7 and about 4.3 or of between about 3.8 and about 4.2 or of between about 3.9 and about 4.1. In one embodiment, the liquid formulation has a pH of about 4.0.

In one embodiment, the liquid formulation further comprises a surfactant. In one embodiment, the surfactant is selected from the group consisting of polysorbates, poloxamers, polyoxyethylenealkylethers, alkylphenolpolyoxyethylene and sodium dodecyl sulfate.

In one embodiment, the concentration of the surfactant in the liquid formulation is equal to or lower than about 0.1% (w/v) or equal to or lower than about 0.05% (w/v).

In one embodiment, the liquid formulation does not comprise any surfactant.

In one embodiment, the liquid formulation does not comprise NaCl.

In one embodiment, the liquid formulation does not comprise any non-buffering salt.

### BRIEF DESCRIPTION OF THE FIGURES

FIG. 1 shows results of the analysis of the stability of two antibodies (mAb1 and mAb3) in formulations containing different buffering systems for pulmonary delivery upon nebulization by dynamic light scattering (DLS; A and D), SEC (B and E), flow cell microscopy (FCM; C and F) and visual inspection (B and E).

FIG. 2 shows results of the analysis of the stability of two antibodies (mAb1 and mAb3) in formulations containing PS80 for pulmonary delivery upon nebulization by DLS (A and D), SEC (B and E), FCM (C and F) and visual inspection (B and E).

FIG. 3 shows results of the analysis of the stability of an antibody (mAb1) in formulations for pulmonary delivery upon nebulization with two different mesh nebulizers by DLS (A), SEC (B), FCM (C) and visual inspection (B).

FIG. 4 shows results of the analysis of the stability of antibodies of different isotypes (IgG1 and IgG4) in formulations for pulmonary delivery upon nebulization by DLS (A), SEC (B), FCM (C) and visual inspection (B).

FIG. 5 shows results of the analysis of the stability of an antibody (mAb1) in a formulation for pulmonary delivery upon nebulization by DLS and FCM (A), SEC and visual inspection (B) as well as by additional analytical methods (B).

FIG. 6 shows results of the analysis of the stability of two antibodies (mAb1 and mAb3) in a formulation containing PS80 in different concentrations in citrate buffer for pulmonary delivery upon nebulization by DLS and FCM. FIG. 6A shows results for mAb1 and FIG. 6B shows results for mAb3.

FIG. 7 shows results of the analysis of the stability of an antibody (mAb1) in a formulation containing PS80 in different concentrations in histidine buffer for pulmonary delivery upon nebulization by DLS and FCM.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. Preferably, the terms used herein are defined as described in “A multilingual glossary of biotechnological terms: (IUPAC Recommendations)”, Leuenberger, H. G. W, Nagel, B. and Köbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments; however, it should be understood that they may be combined in any manner and in any number to create additional embodiments. The variously described examples and preferred/particular embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step. As used in this specification and the appended claims, the singular forms “a”, “an”, and “the” include plural referents, unless the content clearly dictates otherwise.

In one aspect, the present invention relates to an aerosol comprising droplets comprising a liquid formulation, wherein the liquid formulation comprises
(i) an antibody or antibody derivative,
(ii) a buffering agent selected from the group consisting of acetate, histidine and combinations thereof, and
(iii) an aqueous medium; and

wherein the liquid formulation has a pH which is equal to or lower than about 5.5.

In one embodiment, the antibody or antibody derivative is an IgG1 antibody or antibody derivative.

The term “antibody” (or “immunoglobulin”) generally refers to a glycoprotein comprising at least two heavy (H) chains and two light (L) chains inter-connected by disulfide bonds. The term “antibody” includes monoclonal antibodies, recombinant antibodies, human antibodies, humanized antibodies, chimeric antibodies and combinations of any of the foregoing. Each heavy chain is comprised of a heavy chain variable region (VH) and a heavy chain constant region (CH).

Each light chain is comprised of a light chain variable region (VL) and a light chain constant region (CL). The variable regions and constant regions are also referred to herein as variable domains and constant domains, respectively. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The CDRs of a VH are termed HCDR1, HCDR2 and HCDR3, the CDRs of a VL are termed LCDR1, LCDR2 and LCDR3. The variable regions of the heavy and light chains contain a binding domain that interacts with an antigen. The constant regions of an antibody comprise the heavy chain constant region (CH) and the light chain constant region (CL), wherein CH can be further subdivided into constant domain CH1, a hinge region, and constant domains CH2 and CH3 (arranged from amino-terminus to carboxy-terminus in the following order: CH1, CH2, CH3). The constant regions of the antibodies may mediate the binding of the immunoglobulin to host tissues or factors, including various cells of the immune system (e.g., effector cells) and the first component (Clq) of the classical complement system.

Antibodies may be derived from different species, including but not limited to mouse, rat, rabbit, guinea pig and human.

Antibodies described herein include antibodies of the isotypes/classes IgA, such as IgA1 or IgA2, IgG1, IgG2, IgG3, IgG4, IgE, IgM, and IgD. In various embodiments, the antibody is an IgG1 antibody, more particularly an IgG1 kappa or IgG1 lambda isotype (i.e. IgG1, κ, λ), an IgG2a antibody (e.g. IgG2a, κ, λ), an IgG2b antibody (e.g. IgG2b, κ, Δ), an IgG3 antibody (e.g. IgG3, κ, Δ) or an IgG4 antibody (e.g. IgG4, κ, Δ). In one embodiment, the antibody is an IgG1 antibody.

The term “antibody derivative”, as used herein, refers to a molecule comprising at least the domains it is specified to comprise, but not having the overall structure of an antibody such as IgA, IgD, IgE, IgG, IgM, IgY or IgW, in particular IgG1, although still being capable of binding a target molecule. Said derivatives may be, but are not limited to functional (i.e. target binding, particularly specific target binding) antibody fragments thereof, such as Fab2, or combinations of such derivatives, for example bivalent Fabs. It also relates to an antibody to which further antibody domains have been added, such as further variable domains. In one embodiment, the term “antibody derivative” refers to a single chain antibody, e.g., as described in Spiess et al. (J Mol Imm. 2015, 67:95-106), WO 2009/052081 A2, Wu et al. (Nat. Biotechnol., 2007 25:1290-1297), Brinkmann & Kontermann (MAbs 2017, 9:182-212) and Fitzgerald et al. (Mol Cancer Ther. 2013, 13:410-25). Other non-limiting examples of antibody derivatives include nanobodies, diabodies minibodies and other formats as described, e.g., in Spiess et al. (J Mol Imm. 2015, 67:95-106). The term “IgG1 antibody derivative” refers to an antibody derivative which is recognizable as being derived from an IgG1 antibody, e.g., due to the presence of one or more IgG1-specific (sequence-)elements.

In one embodiment, the antibody is a monoclonal antibody. The term “monoclonal antibody”, as used herein, refers to a preparation of antibody molecules of single molecular composition. A monoclonal antibody displays a single binding specificity and affinity. In one embodiment, the monoclonal antibodies are produced by a hybridoma which includes a B-cell obtained from a non-human animal, e.g., a mouse, fused to an immortalized cell.

Generally, the terms “antibody” and “antibody derivative”, as used herein, include both monospecific and multispecific (e.g., bispecific, trispecific or tetraspecific) antibodies and antibody derivatives.

In one embodiment, the antibody or antibody derivative is a pharmaceutically active antibody or antibody derivative. The term “pharmaceutically active antibody or antibody derivative”, as used herein, refers to an antibody or antibody derivative which is suitable for therapy, i.e., can be used to treat a disease or disorder. In one embodiment, the pharmaceutically active antibody or antibody derivative binds to a therapeutically relevant antigen.

Exemplary antibodies that can be used in accordance with the present invention include, but are not limited to, an anti-respiratory syncytial virus (RSV) antibody, an anti-IL-4 antibody, an anti-IL-4 receptor antibody, an anti-IFN-beta antibody, an anti-IL-13 antibody, an anti-thymic stromal lymphopoietin (TSLP) antibody, an anti-IL-1 receptor antibody, an anti-IL-2 antibody and an anti-TNF antibody.

In one embodiment the antibody concentration ranges from about 150 mg/ml to 1 mg/ml; or from about 150 mg/ml to about 5 mg/ml; or from about 150 mg/ml to about 10 mg/ml; or from about 150 mg/ml to about 20 mg/ml; or from about 150 mg/ml to about 50 mg/ml.

In one embodiment the antibody concentration ranges from about 120 mg/ml to 1 mg/ml; or from about 120 mg/ml to about 5 mg/ml; or from about 120 mg/ml to about 10 mg/ml; or from about 120 mg/ml to about 20 mg/ml; or from about 120 mg/ml to about 50 mg/ml.

In one embodiment the antibody concentration ranges from about 100 mg/ml to 1 mg/ml; or from about 100 mg/ml to about 5 mg/ml; or from about 100 mg/ml to about 10 mg/ml; or from about 100 mg/ml to about 20 mg/ml; or from about 100 mg/ml to about 50 mg/ml.

In one embodiment the antibody concentration ranges from about 80 mg/ml to 1 mg/ml; or from about 80 mg/ml to about 5 mg/ml; or from about 80 mg/ml to about 10 mg/ml; or from about 80 mg/ml to about 20 mg/ml; or from about 80 mg/ml to about 50 mg/ml.

In one embodiment the antibody concentration ranges from about 50 mg/ml to 5 mg/ml; or from about 4 Omg/ml to about 5 mg/ml; or from about 30 mg/ml to about 5 mg/ml; or from about 20 mg/ml to about 5 mg/ml; or from about 10 mg/ml to about 5 mg/ml.

In one embodiment the antibody concentration ranges from about 50 mg/ml to 1 mg/ml; or from about 40 mg/ml to about 1 mg/ml; or from about 30 mg/ml to about 1 mg/ml; or from about 20 mg/ml to about 1 mg/ml; or from about 10 mg/ml to about 1 mg/ml.

In a preferred embodiment the concentration of the antibody is about 150 mg/ml. In another preferred embodiment the concentration of the antibody is about 140 mg/ml. In another preferred embodiment the concentration of the antibody is about 130 mg/ml. In another preferred embodiment the concentration of the antibody is about 120 mg/ml. In another preferred embodiment the concentration of the antibody is about 110 mg/ml. In a preferred embodiment the concentration of the antibody is about 100 mg/ml. In another preferred embodiment the concentration of the antibody is about 90 mg/ml. In another preferred embodiment the concentration of the antibody is about 80 mg/ml. In another preferred embodiment the concentration of the antibody is about 70 mg/ml. In another preferred embodiment the concentration of the antibody is about 60 mg/ml. In a preferred embodiment the concentration of the antibody is about 50 mg/ml. In another preferred embodiment the concentration of the antibody is about 40 mg/ml. In another preferred embodiment the concentration of the antibody is about 30 mg/ml. In another preferred embodiment the concentration of the antibody is about 20 mg/ml. In another preferred embodiment the concentration of the antibody is about 10 mg/ml.

In another preferred embodiment the concentration of the antibody is about 5 mg/ml. In another preferred embodiment the concentration of the antibody is about 1 mg/ml.

In one embodiment, the concentration of the buffering agent in the liquid formulation is in the range of from about 1 mM to about 200 mM, e.g., from about 5 mM to about 150 mM or from about 5 mM to about 100 mM or from about 5 mM to about 50 mM. In one embodiment, the concentration of the buffering agent in the liquid formulation is in the range of from about 5 mM to about 25 mM, e.g., from about 5 mM to about 20 mM or from about 5 mM to about 15 mM or from about 7.5 mM to about 12.5 mM. In one embodiment, the concentration of the buffering agent in the liquid formulation is about 10 mM. In one embodiment, in which the buffering agent is acetate and the concentration of acetate in the liquid formulation is in the range of from about 1 mM to about 200 mM, e.g., from about 5 mM to about 150 mM or from about 5 mM to about 100 mM or from about 5 mM to about 50 mM. In one embodiment, the concentration of the acetate in the liquid formulation is in the range of from about 5 mM to about 25 mM, e.g., from about 5 mM to about 20 mM or from about 5 mM to about 15 mM or from about 7.5 mM to about 12.5 mM. In one embodiment, the concentration of the acetate in the liquid formulation is about 10 mM.

In one embodiment, in which the buffering agent is histidine the concentration of the histidine in the liquid formulation is in the range of from about 1 mM to about 200 mM, e.g., from about 5 mM to about 150 mM or from about 5 mM to about 100 mM or from about 5 mM to about 50 mM. In one embodiment, the concentration of the histidine in the liquid formulation is in the range of from about 5 mM to about 25 mM, e.g., from about 5 mM to about 20 mM or from about 5 mM to about 15 mM or from about 7.5 mM to about 12.5 mM. In one embodiment, the concentration of the histidine in the liquid formulation is about 10 mM. In one embodiment, the concentration of the histidine in the liquid formulation is about 20 mM.

In one embodiment, the acetate acting as buffering agent is sodium acetate (or another suitable acetate salt, e.g., potassium acetate), e.g., in conjunction with acetic acid (i.e., in the form of an acetate buffer). Methods to prepare a suitable acetate buffer are well known to a person skilled in the art.

In one embodiment, the histidine acting as buffering agent is L-histidine sodium (or another suitable histidine salt), e.g., in the form of a histidine buffer. Methods to prepare a suitable histidine buffer are well known to a person skilled in the art.

In one embodiment, the liquid formulation does not comprise citrate.

The term “aqueous medium” (or “aqueous solution”), as used herein, refers to a liquid medium or solution in which water is the solvent. In one embodiment, the aqueous medium is/consists of water, in particular purified water or water for injection (WFI). In one embodiment, the aqueous medium is sterile. In one embodiment, the liquid formulation is sterile.

In one embodiment, the liquid formulation has a pH in the range of from about 3.5 to about 5.5.

In one embodiment, the buffering agent is acetate, and the liquid formulation has a pH which is lower than about 5.0 or lower than about 4.5. In one embodiment, the buffering agent is acetate, and the liquid formulation has a pH which is in the range of between about 3.5 and lower than about 4.5 or of between about 3.7 and about 4.3 or of between about 3.8 and about 4.2 or of between about 3.9 and about 4.1. In one embodiment, the liquid formulation has a pH of about 4.0.

In one embodiment, the buffering agent is acetate at a concentration of 10 mM, and the liquid formulation has a pH which is lower than about 5.0 or lower than about 4.5. In one embodiment, the buffering agent is acetate at a concentration of 10 mM, and the liquid formulation has a pH which is in the range of between about 3.5 and lower than about 4.5 or of between about 3.7 and about 4.3 or of between about 3.8 and about 4.2 or of between about 3.9 and about 4.1. In one embodiment, the liquid formulation has a pH of about 4.0 and the buffering agent is acetate at a concentration of 10 mM.

In one embodiment, the buffering agent is histidine, and the liquid formulation has a pH which is equal to or lower than about 5,5. In one embodiment, the buffering agent is histidine, and the liquid formulation has a pH which is lower than about 5.0 or lower than about 4.5. In one embodiment, the buffering agent is histidine, and the liquid formulation has a pH which is in the range of between about 3.5 and lower than about 4.5 or of between about 3.7 and about 4.3 or of between about 3.8 and about 4.2 or of between about 3.9 and about 4.1. In one embodiment, the liquid formulation has a pH of about 4.0.

In one embodiment, the buffering agent is histidine at a concentration of 10 mM, and the liquid formulation has a pH which is equal to or lower than about 5,5. In one embodiment, the buffering agent is histidine at a concentration of 10 mM, and the liquid formulation has a pH which is lower than about 5.0 or lower than about 4.5. In one embodiment, the buffering agent is histidine at a concentration of 10 mM, and the liquid formulation has a pH which is in the range of between about 3.5 and lower than about 4.5 or of between about 3.7 and about 4.3 or of between about 3.8 and about 4.2 or of between about 3.9 and about 4.1. In one embodiment, the liquid formulation has a pH of about 4.0 and the buffering agent is histidine at a concentration of 10 mM.

In one embodiment, the buffering agent is histidine at a concentration of 20 mM, and the liquid formulation has a pH which is equal to or lower than about 5,5. In one embodiment, the buffering agent is histidine at a concentration of 20 mM, and the liquid formulation has a pH which is lower than about 5.0 or lower than about 4.5. In one embodiment, the buffering agent is histidine at a concentration of 20 mM, and the liquid formulation has a pH which is in the range of between about 3.5 and lower than about 4.5 or of between about 3.7 and about 4.3 or of between about 3.8 and about 4.2 or of between about 3.9 and about 4.1. In one embodiment, the liquid formulation has a pH of about 4.0 and the buffering agent is histidine at a concentration of 20 mM.

The liquid formulation may comprise one or more other excipients as long as they are pharmaceutically acceptable and do not compromise the liquid formulation's suitability for administration by inhalation, in particular inhalation via a nebulizer. Suitable excipients are listed in a pharmacopoeia or in, e.g., REMINGTON's PHARMACEUTICAL SCIENCES (18th Ed., A. R. Gennaro, ed., Mack Publishing Company 1990), and subsequent editions of the same). The term “pharmaceutically acceptable”, as used herein, refers to the non-toxicity of a material, which, in one embodiment, does not interact with the action of the active agent of the liquid formulation.

In one embodiment, the liquid formulation further comprises a surfactant.

The term “surfactant” (or “surface active agent”), as used herein, refers to compounds that lower the surface tension (or interfacial tension) between two liquids, between a gas and a liquid, or between a liquid and a solid. In one embodiment, the compounds lower the surface tension (or interfacial tension) between a gas (e.g., air) and a liquid. In one embodiment, the surfactant is a non-ionic surfactant. In one embodiment, the surfactant is selected from the group consisting of polysorbates (e.g., polysorbate 20 or polysorbate 80), poloxamers (e.g., poloxamer 188, commercially available as Pluronic® F68, or poloxamer 407, commercially available as Pluronic® F127), polyoxyethylenealkylethers (e.g., Brij™ surfactants), alkylphenolpolyoxyethylene (e.g., Triton® X100) and sodium dodecyl sulfate (SDS). In one embodiment, the surfactant is polysorbate 80 (PS80).

In one embodiment, the concentration of the surfactant in the liquid formulation is equal to or lower than about 0.1% (w/v) or equal to or lower than about 0.05% (w/v), e.g., equal to or lower than about 0.04% (w/v) or equal to or lower than about 0.03% (w/v) or equal to or lower than about 0.02% (w/v) or equal to or lower than about 0.01% (w/v). In one embodiment, the concentration of the surfactant in the liquid formulation is lower than about 0.01% (w/v).

In one embodiment, the concentration of the polysorbate in the liquid formulation is equal to or lower than about 0.1% (w/v) or equal to or lower than about 0.05% (w/v), e.g., equal to or lower than about 0.04% (w/v) or equal to or lower than about 0.03% (w/v) or equal to or lower than about 0.02% (w/v) or equal to or lower than about 0.01% (w/v). In one embodiment, the concentration of polysorbate in the liquid formulation is lower than about 0.01% (w/v).

In one embodiment, the concentration of the PS80 in the liquid formulation is equal to or lower than about 0.1% (w/v) or equal to or lower than about 0.05% (w/v), e.g., equal to or lower than about 0.04% (w/v) or equal to or lower than about 0.03% (w/v) or equal to or lower than about 0.02% (w/v) or equal to or lower than about 0.01% (w/v). In one embodiment, the concentration of PS80 in the liquid formulation is lower than about 0.01% (w/v).

In one embodiment the liquid formulation only comprises one surfactant. In one embodiment this surfactant is PS80.

In one embodiment, the liquid formulation does not comprise any surfactant, e.g., a surfactant as defined above.

In one embodiment, the liquid formulation does not comprise NaCl.

In one embodiment, the liquid formulation does not comprise any non-buffering salt. The term “non-buffering salt”, as used herein, refers to a salt that does not or not substantially contribute to retaining the pH of the liquid formulation upon addition of an acid or a base. In one embodiment, the non-buffering salt is a halogen salt (e.g., comprising Cl⁻ or Br⁻). In one embodiment, the non-buffering salt is a halogen salt that comprises one or more cations of sodium (Na⁺), potassium (K⁺), calcium (Ca²⁺) or magnesium (Mg²⁺). In one embodiment, the non-buffering salt is a halogen salt that comprises one or more cations of sodium (Na⁺) or potassium (K⁺). In yet another embodiment, the non-buffering salt is selected from the group consisting of NaCl, KCl, CaCl₂ and MgCl₂.

Generally, an aerosol is a suspension of fine solid particles or liquid droplets, in air or another gas. According to the present invention, the term “aerosol” refers to a suspension of droplets of the liquid formulation as defined above in a gas, e.g., air.

In one embodiment the droplets have an average diameter below 5 μm. In one embodiment the droplets have an average diameter below 4.5 μm. In one embodiment the droplets have an average diameter below 4.0 μm. In one embodiment the droplets have an average diameter below 3.5 μm. In one embodiment the droplets have an average diameter below 3.0 μm.

In one embodiment, the droplets have an average diameter in the range of from about 0.5 μm to about 5 μm. In one embodiment, the droplets have an average diameter in the range of from about 0.5 μm to about 4.5 μm. In one embodiment, the droplets have an average diameter in the range of from about 0.5 μm to about 4 μm. In one embodiment, the droplets have an average diameter in the range of from about 0.5 μm to about 3.5 μm. In one embodiment, the droplets have an average diameter in the range of from about 0.5 μm to about 3 μm. In one embodiment, the average diameter is the volume median diameter (VMD; also referred to as Dv50 value). In one embodiment, the VMD is determined by laser diffraction, e.g., as described in U.S.

Pharmacopeia (USP) 429. Droplet size can also be measured by for example interferometric laser imaging. Results may vary upon the measurement method used.

In one embodiment the liquid formulation comprises acetate buffer as buffering agent, has a pH of about 5.0 or lower than about 4.5 and comprises droplets which have an average diameter below 5.0 μm. In one embodiment the liquid formulation comprises acetate buffer as buffering agent, has a pH of about 5.0 or lower than about 4.5 and comprises droplets which have an average diameter in the range of from about 0.5 μm to about 5 μm. In one embodiment the liquid formulation comprises acetate buffer as buffering agent, has a pH in the range of between about 3.5 and lower than about 4.5 and comprises droplets which have an average diameter below 5 μm. In one embodiment the liquid formulation comprises acetate buffer as buffering agent, has a pH in the range of between about 3.5 and lower than about 4.5 and comprises droplets which have an average diameter in the range of from about 0.5 μm to about 5 μm. In one embodiment, the liquid formulation comprises acetate buffer as buffering agent, has a pH which is or of between about 3.7 and about 4.3 and comprises droplets which have an average diameter below 5 μm. In one embodiment, the liquid formulation comprises acetate buffer as buffering agent, has a pH which is or of between about 3.7 and about 4.3 and comprises droplets which have an average diameter in the range of from about 0.5 μm to about 5 μm. In one embodiment, the liquid formulation comprises acetate buffer as buffering agent, has a pH which is or of between about 3.8 and about 4.2 and comprises droplets which have an average diameter below 5 μm. In one embodiment, the liquid formulation comprises acetate buffer as buffering agent, has a pH which is or of between about 3.8 and about 4.2 and comprises droplets which have an average diameter in the range of from about 0.5 μm to about 5 μm. In one embodiment, the liquid formulation comprises acetate buffer as buffering agent, has a pH which is or of between about 3.9 and about 4.1 and comprises droplets which have an average diameter below 5 μm. In one embodiment, the liquid formulation comprises acetate buffer as buffering agent, has a pH which is or of between about 3.9 and about 4.1 and comprises droplets which have an average diameter in the range of from about 0.5 μm to about 5 μm. In another embodiment the liquid formulation only comprises one surfactant. In one embodiment this surfactant is PS80.

In one embodiment the liquid formulation comprises acetate buffer as buffering agent, has a pH of about 5.0 or lower than about 4.5 and comprises droplets which have an average diameter below 4.5 μm. In one embodiment the liquid formulation comprises acetate buffer as buffering agent, has a pH of about 5.0 or lower than about 4.5 and comprises droplets which have an average diameter in the range of from about 0.5 μm to about 4.5 μm. In one embodiment the liquid formulation comprises acetate buffer as buffering agent, has a pH in the range of between about 3.5 and lower than about 4.5 and comprises droplets which have an average diameter below 4.5 μm. In one embodiment the liquid formulation comprises acetate buffer as buffering agent, has a pH in the range of between about 3.5 and lower than about 4.5 and comprises droplets which have an average diameter in the range of from about 0.5 μm to about 4.5 μm. In one embodiment, the liquid formulation comprises acetate buffer as buffering agent, has a pH which is or of between about 3.7 and about 4.3 and comprises droplets which have an average diameter below 4.5 μm. In one embodiment, the liquid formulation comprises acetate buffer as buffering agent, has a pH which is or of between about 3.7 and about 4.3 and comprises droplets which have an average diameter in the range of from about 0.5 μm to about 4.5 μm. In one embodiment, the liquid formulation comprises acetate buffer as buffering agent, has a pH which is or of between about 3.8 and about 4.2 and comprises droplets which have an average diameter below 4.5 μm. In one embodiment, the liquid formulation comprises acetate buffer as buffering agent, has a pH which is or of between about 3.8 and about 4.2 and comprises droplets which have an average diameter in the range of from about 0.5 μm to about 4.5 μm. In one embodiment, the liquid formulation comprises acetate buffer as buffering agent, has a pH which is or of between about 3.9 and about 4.1 and comprises droplets which have an average diameter below 4.5 μm. In one embodiment, the liquid formulation comprises acetate buffer as buffering agent, has a pH which is or of between about 3.9 and about 4.1 and comprises droplets which have an average diameter in the range of from about 0.5 μm to about 4.5 μm. In another embodiment the liquid formulation only comprises one surfactant. In one embodiment this surfactant is PS80.

In one embodiment the liquid formulation comprises acetate buffer as buffering agent, has a pH of about 5.0 or lower than about 4.5 and comprises droplets which have an average below 4 μm.

In one embodiment the liquid formulation comprises acetate buffer as buffering agent, has a pH of about 5.0 or lower than about 4.5 and comprises droplets which have an average diameter in the range of from about 0.5 μm to about 4 μm. In one embodiment the liquid formulation comprises acetate buffer as buffering agent, has a pH in the range of between about 3.5 and lower than about 4.5 and comprises droplets which have an average diameter below 4 μm. In one embodiment the liquid formulation comprises acetate buffer as buffering agent, has a pH in the range of between about 3.5 and lower than about 4.5 and comprises droplets which have an average diameter in the range of from about 0.5 μm to about 4 μm. In one embodiment, the liquid formulation comprises acetate buffer as buffering agent, has a pH which is or of between about 3.7 and about 4.3 and comprises droplets which have an average diameter below 4 μm. In one embodiment, the liquid formulation comprises acetate buffer as buffering agent, has a pH which is or of between about 3.7 and about 4.3 and comprises droplets which have an average diameter in the range of from about 0.5 μm to about 4 μm. In one embodiment, the liquid formulation comprises acetate buffer as buffering agent, has a pH which is or of between about 3.8 and about 4.2 and comprises droplets which have an average diameter below 4 μm. In one embodiment, the liquid formulation comprises acetate buffer as buffering agent, has a pH which is or of between about 3.8 and about 4.2 and comprises droplets which have an average diameter in the range of from about 0.5 μm to about 4 μm. In one embodiment, the liquid formulation comprises acetate buffer as buffering agent, has a pH which is or of between about 3.9 and about 4.1 and comprises droplets which have an average diameter below 4 μm. In one embodiment, the liquid formulation comprises acetate buffer as buffering agent, has a pH which is or of between about 3.9 and about 4.1 and comprises droplets which have an average diameter in the range of from about 0.5 μm to about 4 μm. In another embodiment the liquid formulation only comprises one surfactant. In one embodiment this surfactant is PS80.

In one embodiment the liquid formulation comprises acetate buffer as buffering agent, has a pH of about 5.0 or lower than about 4.5 and comprises droplets which have an average diameter below 3.5 μm. In one embodiment the liquid formulation comprises acetate buffer as buffering agent, has a pH of about 5.0 or lower than about 4.5 and comprises droplets which have an average diameter in the range of from about 0.5 μm to about 3.5 μm. In one embodiment the liquid formulation comprises acetate buffer as buffering agent, has a pH in the range of between about 3.5 and lower than about 4.5 and comprises droplets which have an average diameter below 3.5 μm. In one embodiment the liquid formulation comprises acetate buffer as buffering agent, has a pH in the range of between about 3.5 and lower than about 4.5 and comprises droplets which have an average diameter in the range of from about 0.5 μm to about 3.5 μm. In one embodiment, the liquid formulation comprises acetate buffer as buffering agent, has a pH which is or of between about 3.7 and about 4.3 and comprises droplets which have an average diameter below 3.5 μm. In one embodiment, the liquid formulation comprises acetate buffer as buffering agent, has a pH which is or of between about 3.7 and about 4.3 and comprises droplets which have an average diameter in the range of from about 0.5 μm to about 3.5 μm. In one embodiment, the liquid formulation comprises acetate buffer as buffering agent, has a pH which is or of between about 3.8 and about 4.2 and comprises droplets which have an average diameter below 3.5 μm. In one embodiment, the liquid formulation comprises acetate buffer as buffering agent, has a pH which is or of between about 3.8 and about 4.2 and comprises droplets which have an average diameter in the range of from about 0.5 μm to about 3.5 μm. In one embodiment, the liquid formulation comprises acetate buffer as buffering agent, has a pH which is or of between about 3.9 and about 4.1 and comprises droplets which have an average diameter below 3.5 μm. In one embodiment, the liquid formulation comprises acetate buffer as buffering agent, has a pH which is or of between about 3.9 and about 4.1 and comprises droplets which have an average diameter in the range of from about 0.5 μm to about 3.5 μm. In another embodiment the liquid formulation only comprises one surfactant. In one embodiment this surfactant is PS80.

In one embodiment the liquid formulation comprises acetate buffer as buffering agent, has a pH of about 5.0 or lower than about 4.5 and comprises droplets which have an average diameter below 3 μm. In one embodiment the liquid formulation comprises acetate buffer as buffering agent, has a pH of about 5.0 or lower than about 4.5 and comprises droplets which have an average diameter in the range of from about 0.5 μm to about 3 μm. In one embodiment the liquid formulation comprises acetate buffer as buffering agent, has a pH in the range of between about 3.5 and lower than about 4.5 and comprises droplets which have an average diameter below 3 μm. In one embodiment the liquid formulation comprises acetate buffer as buffering agent, has a pH in the range of between about 3.5 and lower than about 4.5 and comprises droplets which have an average diameter in the range of from about 0.5 μm to about 3 μm. In one embodiment, the liquid formulation comprises acetate buffer as buffering agent, has a pH which is or of between about 3.7 and about 4.3 and comprises droplets which have an average diameter below 3 μm. In one embodiment, the liquid formulation comprises acetate buffer as buffering agent, has a pH which is or of between about 3.7 and about 4.3 and comprises droplets which have an average diameter in the range of from about 0.5 μm to about 3 μm. In one embodiment, the liquid formulation comprises acetate buffer as buffering agent, has a pH which is or of between about 3.8 and about 4.2 and comprises droplets which have an average diameter below 3 μm. In one embodiment, the liquid formulation comprises acetate buffer as buffering agent, has a pH which is or of between about 3.8 and about 4.2 and comprises droplets which have an average diameter in the range of from about 0.5 μm to about 3 μm. In one embodiment, the liquid formulation comprises acetate buffer as buffering agent, has a pH which is or of between about 3.9 and about 4.1 and comprises droplets which have an average diameter below 3 μm. In one embodiment, the liquid formulation comprises acetate buffer as buffering agent, has a pH which is or of between about 3.9 and about 4.1 and comprises droplets which have an average diameter in the range of from about 0.5 μm to about 3 μm. In another embodiment the liquid formulation only comprises one surfactant. In one embodiment this surfactant is PS80.

In one embodiment the liquid formulation comprises histidine buffer as buffering agent, has a pH of about 5.0 or lower than about 4.5 and comprises droplets which have an average diameter below 5 μm. In one embodiment the liquid formulation comprises histidine buffer as buffering agent, has a pH of about 5.0 or lower than about 4.5 and comprises droplets which have an average diameter in the range of from about 0.5 μm to about 5 μm. In one embodiment the liquid formulation comprises histidine buffer as buffering agent, has a pH in the range of between about 3.5 and lower than about 4.5 and comprises droplets which have an average diameter below 5 μm. In one embodiment the liquid formulation comprises histidine buffer as buffering agent, has a pH in the range of between about 3.5 and lower than about 4.5 and comprises droplets which have an average diameter in the range of from about 0.5 μm to about 5 μm. In one embodiment, the liquid formulation comprises histidine buffer as buffering agent, has a pH which is or of between about 3.7 and about 4.3 and comprises droplets which have an average diameter below 5 μm. In one embodiment, the liquid formulation comprises histidine buffer as buffering agent, has a pH which is or of between about 3.7 and about 4.3 and comprises droplets which have an average diameter in the range of from about 0.5 μm to about 5 μm. In one embodiment, the liquid formulation comprises histidine buffer as buffering agent, has a pH which is or of between about 3.8 and about 4.2 and comprises droplets which have an average diameter below 5 μm. In one embodiment, the liquid formulation comprises histidine buffer as buffering agent, has a pH which is or of between about 3.8 and about 4.2 and comprises droplets which have an average diameter in the range of from about 0.5 μm to about 5 μm. In one embodiment, the liquid formulation comprises histidine buffer as buffering agent, has a pH which is or of between about 3.9 and about 4.1 and comprises droplets which have an average diameter below 5 μm. In one embodiment, the liquid formulation comprises histidine buffer as buffering agent, has a pH which is or of between about 3.9 and about 4.1 and comprises droplets which have an average diameter in the range of from about 0.5 μm to about 5 μm. In another embodiment the liquid formulation only comprises one surfactant. In one embodiment this surfactant is PS80.

In one embodiment the liquid formulation comprises histidine buffer as buffering agent, has a pH of about 5.0 or lower than about 4.5 and comprises droplets which have an average diameter below 4.5 μm. In one embodiment the liquid formulation comprises histidine buffer as buffering agent, has a pH of about 5.0 or lower than about 4.5 and comprises droplets which have an average diameter in the range of from about 0.5 μm to about 4.5 μm. In one embodiment the liquid formulation comprises histidine buffer as buffering agent, has a pH in the range of between about 3.5 and lower than about 4.5 and comprises droplets which have an average diameter below 4.5 μm. In one embodiment the liquid formulation comprises histidine buffer as buffering agent, has a pH in the range of between about 3.5 and lower than about 4.5 and comprises droplets which have an average diameter in the range of from about 0.5 μm to about 4.5 μm. In one embodiment, the liquid formulation comprises histidine buffer as buffering agent, has a pH which is or of between about 3.7 and about 4.3 and comprises droplets which have an average diameter below 4.5 μm. In one embodiment, the liquid formulation comprises histidine buffer as buffering agent, has a pH which is or of between about 3.7 and about 4.3 and comprises droplets which have an average diameter in the range of from about 0.5 μm to about 4.5 μm. In one embodiment, the liquid formulation comprises acetate buffer as buffering agent, has a pH which is or of between about 3.8 and about 4.2 and comprises droplets which have an average diameter below 4.5 μm. In one embodiment, the liquid formulation comprises acetate buffer as buffering agent, has a pH which is or of between about 3.8 and about 4.2 and comprises droplets which have an average diameter in the range of from about 0.5 μm to about 4.5 μm. In one embodiment, the liquid formulation comprises histidine buffer as buffering agent, has a pH which is or of between about 3.9 and about 4.1 and comprises droplets which have an average diameter below 4.5 μm. In one embodiment, the liquid formulation comprises histidine buffer as buffering agent, has a pH which is or of between about 3.9 and about 4.1 and comprises droplets which have an average diameter in the range of from about 0.5 μm to about 4.5 μm. In another embodiment the liquid formulation only comprises one surfactant. In one embodiment this surfactant is PS80.

In one embodiment the liquid formulation comprises histidine buffer as buffering agent, has a pH of about 5.0 or lower than about 4.5 and comprises droplets which have an average diameter below 4 μm. In one embodiment the liquid formulation comprises histidine buffer as buffering agent, has a pH of about 5.0 or lower than about 4.5 and comprises droplets which have an average diameter in the range of from about 0.5 μm to about 4 μm. In one embodiment the liquid formulation comprises histidine buffer as buffering agent, has a pH in the range of between about 3.5 and lower than about 4.5 and comprises droplets which have an average diameter below 4 μm. In one embodiment the liquid formulation comprises histidine buffer as buffering agent, has a pH in the range of between about 3.5 and lower than about 4.5 and comprises droplets which have an average diameter in the range of from about 0.5 μm to about 4 μm. In one embodiment, the liquid formulation comprises histidine buffer as buffering agent, has a pH which is or of between about 3.7 and about 4.3 and comprises droplets which have an average diameter below 4 μm. In one embodiment, the liquid formulation comprises histidine buffer as buffering agent, has a pH which is or of between about 3.7 and about 4.3 and comprises droplets which have an average diameter in the range of from about 0.5 μm to about 4 μm. In one embodiment, the liquid formulation comprises histidine buffer as buffering agent, has a pH which is or of between about 3.8 and about 4.2 and comprises droplets which have an average diameter below 4 μm. In one embodiment, the liquid formulation comprises histidine buffer as buffering agent, has a pH which is or of between about 3.8 and about 4.2 and comprises droplets which have an average diameter in the range of from about 0.5 μm to about 4 μm. In one embodiment, the liquid formulation comprises histidine buffer as buffering agent, has a pH which is or of between about 3.9 and about 4.1 and comprises droplets which have an average diameter below 4 μm. In one embodiment, the liquid formulation comprises histidine buffer as buffering agent, has a pH which is or of between about 3.9 and about 4.1 and comprises droplets which have an average diameter in the range of from about 0.5 μm to about 4 μm. In another embodiment the liquid formulation only comprises one surfactant. In one embodiment this surfactant is PS80.

In one embodiment the liquid formulation comprises histidine buffer as buffering agent, has a pH of about 5.0 or lower than about 4.5 and comprises droplets which have an average below 3.5 μm. In one embodiment the liquid formulation comprises histidine buffer as buffering agent, has a pH of about 5.0 or lower than about 4.5 and comprises droplets which have an average diameter in the range of from about 0.5 μm to about 3.5 μm. In one embodiment the liquid formulation comprises histidine buffer as buffering agent, has a pH in the range of between about 3.5 and lower than about 4.5 and comprises droplets which have an average diameter below 3.5 μm. In one embodiment the liquid formulation comprises histidine buffer as buffering agent, has a pH in the range of between about 3.5 and lower than about 4.5 and comprises droplets which have an average diameter in the range of from about 0.5 μm to about 3.5 μm. In one embodiment, the liquid formulation comprises histidine buffer as buffering agent, has a pH which is or of between about 3.7 and about 4.3 and comprises droplets which have an average diameter below 3.5 μm.

In one embodiment, the liquid formulation comprises histidine buffer as buffering agent, has a pH which is or of between about 3.7 and about 4.3 and comprises droplets which have an average diameter in the range of from about 0.5 μm to about 3.5 μm. In one embodiment, the liquid formulation comprises histidine buffer as buffering agent, has a pH which is or of between about 3.8 and about 4.2 and comprises droplets which have an average diameter below 3.5 μm. In one embodiment, the liquid formulation comprises histidine buffer as buffering agent, has a pH which is or of between about 3.8 and about 4.2 and comprises droplets which have an average diameter in the range of from about 0.5 μm to about 3.5 μm. In one embodiment, the liquid formulation comprises histidine buffer as buffering agent, has a pH which is or of between about 3.9 and about 4.1 and comprises droplets which have an average diameter below 3.5 μm. In one embodiment, the liquid formulation comprises histidine buffer as buffering agent, has a pH which is or of between about 3.9 and about 4.1 and comprises droplets which have an average diameter in the range of from about 0.5 μm to about 3.5 μm. In another embodiment the liquid formulation only comprises one surfactant. In one embodiment this surfactant is PS80.

In one embodiment the liquid formulation comprises histidine buffer as buffering agent, has a pH of about 5.0 or lower than about 4.5 and comprises droplets which have an average diameter below 3 μm. In one embodiment the liquid formulation comprises histidine buffer as buffering agent, has a pH of about 5.0 or lower than about 4.5 and comprises droplets which have an average diameter in the range of from about 0.5 μm to about 3 μm. In one embodiment the liquid formulation comprises histidine buffer as buffering agent, has a pH in the range of between about 3.5 and lower than about 4.5 and comprises droplets which have an average diameter below 3 μm. In one embodiment the liquid formulation comprises histidine buffer as buffering agent, has a pH in the range of between about 3.5 and lower than about 4.5 and comprises droplets which have an average diameter in the range of from about 0.5 μm to about 3 μm. In one embodiment, the liquid formulation comprises histidine buffer as buffering agent, has a pH which is or of between about 3.7 and about 4.3 and comprises droplets which have an average diameter below 3 μm. In one embodiment, the liquid formulation comprises histidine buffer as buffering agent, has a pH which is or of between about 3.7 and about 4.3 and comprises droplets which have an average diameter in the range of from about 0.5 μm to about 3 μm. In one embodiment, the liquid formulation comprises histidine buffer as buffering agent, has a pH which is or of between about 3.8 and about 4.2 and comprises droplets which have an average diameter below 3 μm. In one embodiment, the liquid formulation comprises histidine buffer as buffering agent, has a pH which is or of between about 3.8 and about 4.2 and comprises droplets which have an average diameter in the range of from about 0.5 μm to about 3 μm. In one embodiment, the liquid formulation comprises histidine buffer as buffering agent, has a pH which is or of between about 3.9 and about 4.1 and comprises droplets which have an average diameter below 3 μm. In one embodiment, the liquid formulation comprises histidine buffer as buffering agent, has a pH which is or of between about 3.9 and about 4.1 and comprises droplets which have an average diameter in the range of from about 0.5 μm to about 3 μm. In another embodiment the liquid formulation only comprises one surfactant. In one embodiment this surfactant is PS80.

According to the present invention, the antibodies or antibody derivatives which are present in the aerosols or liquid formulations described herein are characterized by low aggregation, e.g., as compared to the same antibodies or antibody derivatives being formulated in a formulation comprising citrate.

In one embodiment, the antibodies or antibody derivatives which are present in the aerosols or liquid formulations described herein have one or more of the following properties:
the polydispersity index (PDI) of the antibodies or antibody derivatives which are present in the aerosols or liquid formulations described herein is 0.5 or less, or 0.4 or less, or 0.3 or less, or 0.2 or less, or 0.1 or less, e.g., as determined by DLS (e.g., essentially as described in Example 1);
the percentage of polydispersity of the monomers of the antibodies or antibody derivatives which are present in the aerosols or liquid formulations described herein is 30% or less, or 25% or less, or 20% or less, or 15% or less, e.g., as determined by DLS (e.g., essentially as described in Example 1);
the mass percentage of the monomers of the antibodies or antibody derivatives which are present in the aerosols or liquid formulations described herein is 99.7% or more, or 99.8% or more, or more than 99.8%, e.g., as determined by DLS (e.g., essentially as described in Example 1);
the intensity percentage of the monomers of the antibodies or antibody derivatives which are present in the aerosols or liquid formulations described herein is 80.0% or more, or 85.0% or more, or 90.0% or more, or more than 90.0%, e.g., as determined by DLS (e.g., essentially as described in Example 1);
the number of particles >2 μm is less than 10000 per mL, or less than 7500 per mL, or less than 5000 per mL, or less than 4000 per mL, or less than 3000 per mL, or less than 2000 per mL;
the number of particles >10 μm is less than 500 per mL, or less than 400 per mL, or less than 300 per mL, or less than 200 per mL; and the number of particles >25 μm is less than 100 per mL, or less than 50 per mL, or less than 40 per mL, or less than 30 per mL, or less than 20 per mL, e.g., as determined by FCM (e.g., essentially as described in Example 1).

In another aspect, the present invention relates to a method of preparing an aerosol comprising droplets comprising a liquid formulation, said method comprising the steps:
(i) providing a liquid formulation as defined above,
(ii) nebulizing the liquid formulation provided in step (i) by means of a nebulizer, thereby preparing the aerosol.

In one embodiment, the nebulizer is a mesh nebulizer.

Nebulizers allow the dispersion of a liquid in a gas to aerosolize a liquid formulation into an aerosol that is inhaled into a subject's respiratory tract. Examples of nebulizers include a soft mist nebulizer, a mesh nebulizer (e.g., a vibrating mesh nebulizer), a jet nebulizer and an ultrasonic wave nebulizer. Suitable nebulizer devices include the Aerogen® Solo (Aerogen), Pari eFlow® (Pari GmbH), Philips I-neb™ (Philips), the Pari LC Sprint (Pari GmbH), the AERxR™ Pulmonary Delivery System (Aradigm Corp.) and the Pari LC Plus Reusable Nebulizer (Pari GmbH). In one embodiment, the nebulizer is a mesh nebulizer, in particular a vibrating mesh nebulizer. A nebulizer typically comprises from about 1 mL to about 200 mL, more typically from 1 mL to 20 mL of the liquid formulation.

In one embodiment, the method further comprises, between steps (i) and (ii), the steps of:
(ia) lyophilizing the liquid formulation provided in step (i), thereby providing a lyophilized powder, and
(ib) reconstituting the liquid formulation provided in step (i) by adding an appropriate amount of an aqueous medium to the lyophilized powder provided in step (ia).

In another aspect, the present invention relates to an aerosol comprising droplets comprising a liquid formulation, wherein the aerosol is obtainable by the method as defined above. In one embodiment, the droplets have an average diameter in the range of from about 0.5 μm to about m or from about 0.5 μm to about 3 μm.

In another aspect, the present invention relates to the liquid formulation as defined above or the aerosol as defined above for use in a method of delivering an antibody or antibody derivative to the lungs of a subject, wherein the aerosol is administered to the subject by inhalation or the liquid formulation is administered to the subject by inhalation via a nebulizer.

In one embodiment, the antibody or antibody derivative is an IgG1 antibody or antibody derivative.

In one embodiment, the nebulizer is a mesh nebulizer.

The term “subject” means according to the invention a subject for treatment, in particular a diseased subject (also referred to as “patient”), including human beings, non-human primates or other animals, in particular mammals, such as cows, horses, pigs, sheep, goats, dogs, cats, rabbits or rodents, such as mice, rats, guinea pigs and hamsters. In one embodiment, the subject/patient is a human being.

In another aspect, the present invention relates to the liquid formulation as defined above for use in a method of treating or preventing a disease in a subject, wherein the aerosol is administered to the subject by inhalation or the liquid formulation is administered to the subject by inhalation via a nebulizer.

In one embodiment, the disease is a lung disease.

In one embodiment, the nebulizer is a mesh nebulizer.

The term “treating”, as used herein, relates to any treatment, which improves the health status and/or prolongs (increases) the lifespan of a patient.

According to the invention, the term “disease” refers to any pathological state, in particular cancer, infectious diseases, inflammatory diseases, metabolic diseases, autoimmune disorders, degenerative diseases, apoptosis-associated diseases and transplant rejections.

The term “cancer” according to the invention also comprises cancer metastases. By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor, i.e. a secondary tumor or metastatic tumor, at the target site depends on angiogenesis. Tumor metastasis often occurs even after the removal of the primary tumor because tumor cells or components may remain and develop metastatic potential. In one embodiment, the term “metastasis” according to the invention relates to “distant metastasis” which relates to a metastasis which is remote from the primary tumor and the regional lymph node system.

The term “infectious disease” refers to any disease which can be transmitted from individual to individual or from organism to organism, and is caused by a microbial agent (e.g. common cold). Examples of infectious diseases include viral infectious diseases, such as AIDS (HIV), hepatitis A, B or C, herpes, herpes zoster (chicken-pox), German measles (rubella virus), yellow fever, dengue etc. flaviviruses, influenza viruses, respiratory syncytial virus (RSV), hemorrhagic infectious diseases (Marburg or Ebola viruses), and severe acute respiratory syndrome (SARS), bacterial infectious diseases, such as Legionnaire's disease (Legionella), sexually transmitted diseases (e.g. chlamydia or gonorrhea), gastric ulcer (Helicobacter), cholera (Vibrio), tuberculosis, diphtheria, infections by E. coli, Staphylococci, Salmonella or Streptococci (tetanus); infections by protozoan pathogens such as malaria, sleeping sickness, leishmaniasis; toxoplasmosis, i.e. infections by Plasmodium, Trypanosoma, Leishmania and Toxoplasma; or fungal infections, which are caused, e.g., by Cryptococcus neoformans, Histoplasma capsulatum, Coccidioides immitis, Blastomyces dermatitidis or Candida albicans.

The term “inflammatory disease” refers to any disease, which is characterized by or associated with high levels of inflammation in tissues, in particular connective tissues, or degeneration of these tissues. A chronic inflammatory disease is a medical condition which is characterized by persistent inflammation. Examples of (chronic) inflammatory diseases include celiac disease, vasculitis, lupus, chronic obstructive pulmonary disease (COPD), asthma, irritable bowel disease, atherosclerosis, arthritis, ankylosing spondylitis, Crohn's disease, colitis, chronic active hepatitis, dermatitis and psoriasis.

The term “metabolic disease” refers to any disease or disorder that disrupts normal metabolism. Examples include cystinosis, diabetes, dyslipidemia, hyperthyroidism, hypothyroidism, hyperlipidemia, hypolipidemia, galactosemia, Gaucher's disease, obesity and phenylketonuria.

The term “autoimmune disorder” refers to any disease/disorder in which the body produces an immunogenic (i.e. immune system) response to some constituent of its own tissue. In other words, the immune system loses its ability to recognize some tissue or system within the body as self and targets and attacks it as if it were foreign. Autoimmune diseases can be classified into those in which predominantly one organ is affected (e.g. hemolytic anemia and anti-immune thyroiditis), and those in which the autoimmune disease process is diffused through many tissues (e.g. systemic lupus erythematosus). For example, multiple sclerosis is thought to be caused by T cells attacking the sheaths that surround the nerve fibers of the brain and spinal cord. This results in loss of coordination, weakness, and blurred vision. Autoimmune diseases are known in the art and include, for instance, Hashimoto's thyroiditis, Grave's disease, lupus, multiple sclerosis, rheumatic arthritis, hemolytic anemia, anti-immune thyroiditis, systemic lupus erythematosus, celiac disease, Crohn's disease, colitis, diabetes, scleroderma, psoriasis, and the like.

The term “degenerative disease” refers to any disease in which the function or structure of the affected tissues or organs will increasingly deteriorate over time. Examples include Alzheimer's disease, Parkinson's disease, amyotrophic lateral sclerosis (ALS), Huntington's disease, macular degeneration, multiple sclerosis, muscular dystrophy, Niemann Pick disease, osteoporosis and rheumatoid arthritis.

The term “apoptosis-associated diseases” refers to any disease in which alterations of apoptosis are involved. Examples include cancer, neurological disorders, such as Alzheimer's disease, Parkinson's disease, Huntington's disease, amyotrophic lateral sclerosis (ALS) and stroke, heart diseases, such as ischemia reperfusion and chronic heart failure, infectious diseases and autoimmune diseases.

The term “transplant rejection” refers to the rejection of a transplanted tissue or organ by the recipient's immune system, which may, ultimately, destroy the transplanted tissue or organ.

The term “lung disease” (also referred to as “pulmonary disease” herein) generally refers to diseases or disorder that affect the lungs of a subject. In one embodiment, the lung disease is selected from the group consisting of asthma, chronic obstructive pulmonary disease (COPD), lung cancer (e.g., non-small cell lung cancer), cystic fibrosis (CF), interstitial lung disease (ILD), such as idiopathic pulmonary fibrosis, hypersensitivity pneumonitis, sarcoidosis and asbestosis, bacterial infections (e.g., mycobacterial infection), viral infections (e.g., Influenza virus infection or respiratory syncytial virus (RSV) infection), acute respiratory distress syndrome (ARDS), pulmonary alveolar proteinosis (PAP), acute bronchitis, bronchiolitis obliterans and pulmonary hypertension.

In another aspect, the present invention relates to a method of delivering an antibody or antibody derivative to the lungs of a subject, said method comprising administering to the subject an effective amount of the aerosol as defined above by inhalation or administering to the subject an effective amount of the liquid formulation as defined above by inhalation via a nebulizer.

In one embodiment, the antibody or antibody derivative is an IgG1 antibody or antibody derivative.

In one embodiment, the nebulizer is a mesh nebulizer.

The term “effective amount”, as used herein, refers, in particular, to a “therapeutically effective amount”, which is an amount that achieves a desired therapeutic reaction or a desired therapeutic effect alone or together with further doses, particularly without causing unacceptable side-effects. In the case of treatment of a particular disease or of a particular condition, the desired reaction particularly relates to inhibition of the course of the disease. This comprises slowing down the progress of the disease and, in particular, interrupting or reversing the progress of the disease. The desired reaction in a treatment of a disease or of a condition may also be delay of the onset or a prevention of the onset of said disease or said condition. An effective amount of an aerosol or liquid formulation as described herein, and, thus, of the antibody or antibody derivative contained therein, will depend on the condition to be treated, the severeness of the disease, the individual parameters of the subject, including age, physiological condition, size and weight, the duration of treatment, the type of an accompanying therapy (if present), the specific route of administration and similar factors. Accordingly, the doses administered of the aerosol or liquid formulation described herein may depend on several of such parameters. In the case that a reaction in a subject is insufficient with an initial dose, higher doses may be used.

In another aspect, the present invention relates to a method of treating or preventing a disease in a subject, said method comprising administering to the subject an effective amount of the aerosol as defined above by inhalation or administering to the subject an effective amount of the liquid formulation as defined above by inhalation via a nebulizer.

In one embodiment, the disease is a lung disease.

In one embodiment, the nebulizer is a mesh nebulizer.

In another aspect, the present invention relates to a nebulizer comprising a liquid formulation as defined above.

In one embodiment, the nebulizer is a mesh nebulizer.

In another aspect, the present invention relates to a kit comprising
(i) a container comprising the liquid formulation as defined above or a powder obtainable by lyophilization of the liquid formulation, and
(ii) a nebulizer.

In one embodiment, the nebulizer is a mesh nebulizer.

As used herein, the term “kit of parts (in short: kit)” refers to an article of manufacture comprising one or more containers, a nebulizer (e.g., a mesh nebulizer), and, optionally, a data carrier. Said one or more containers are filled with the liquid formulation as defined above and/or a powder obtainable by lyophilization of the liquid formulation. Additional containers may be included in the kit that contain, e.g., diluents (e.g., an aqueous medium), buffers and further reagents as defined herein. Said data carrier may be a non-electronical data carrier, e.g., a graphical data carrier such as an information leaflet, an information sheet, a bar code or an access code, or an electronical data carrier such as a compact disk (CD), a digital versatile disk (DVD), a microchip or another semiconductor-based electronical data carrier. The access code may allow the access to a database, e.g., an internet database, a centralized, or a decentralized database. Said data carrier may comprise instructions for the use of the kit in the methods and uses as described herein.

In another aspect, the present invention relates to the use of a liquid formulation as defined above for preparing an aerosol by nebulization by means of a nebulizer.

In one embodiment, the nebulizer is a mesh nebulizer.

In another aspect, the present invention relates to the use of a buffering agent selected from the group consisting of acetate, histidine and combinations thereof for increasing the stability of an antibody or antibody derivative upon nebulization of a liquid formulation comprising the antibody or antibody derivative by means of a nebulizer, wherein the buffering agent is included in the liquid formulation prior to nebulization.

In one embodiment, the antibody or antibody derivative is an IgG1 antibody or antibody derivative.

In one embodiment, the nebulizer is a mesh nebulizer.

In one embodiment, the term “increasing the stability” refers to preventing or reducing the extent of aggregation of the antibody or antibody derivative.

In one embodiment, the liquid formulation has a pH which is equal to or lower than about 5.5.

In one embodiment, the antibody is a monoclonal antibody.

In one embodiment, the liquid formulation does not comprise citrate.

In one embodiment, the liquid formulation has a pH in the range of from about 3.5 to about 5.5.

In one embodiment, the buffering agent is acetate, and the liquid formulation has a pH which is lower than about 5.0 or lower than about 4.5. In one embodiment, the buffering agent is acetate, and the liquid formulation has a pH which is in the range of between about 3.5 and lower than about 4.5 or of between about 3.7 and about 4.3 or of between about 3.8 and about 4.2 or of between about 3.9 and about 4.1. In one embodiment, the liquid formulation has a pH of about 4.0.

In one embodiment, the liquid formulation further comprises a surfactant. In one embodiment, the surfactant is selected from the group consisting of polysorbates, poloxamers, polyoxyethylenealkylethers, alkylphenolpolyoxyethylene and sodium dodecyl sulfate.

In one embodiment, the concentration of the surfactant in the liquid formulation is equal to or lower than about 0.1% (w/v) or equal to or lower than about 0.05% (w/v).

In one embodiment, the liquid formulation does not comprise any surfactant.

In one embodiment, the liquid formulation does not comprise NaCl.

In one embodiment, the liquid formulation does not comprise any non-buffering salt.

The present invention is now further described by reference to the following Examples, which are intended to illustrate, not to limit the scope of the present invention.

### EXAMPLES

### Example 1: Material and Methods

The immunoglobulins used in the examples are listed in below Table 1.

TABLE 1
Immunoglobulins.

| Name | Isotype | pl | MW (kDa) |
|---|---|---|---|
| mAb1 | IgG1 | 7.6-8.5 | 145 |
| mAb2 | IgG1 | 8.2-9.1 | 143 |
| mAb3 | IgG1 | 7.0-10.0 | 145 |
| mAb4 | IgG1 | 7.1-8.1 | 147 |
| mAbX | IgG4 | <7.0 | 147 |

### Dynamic Light Scattering (DLS)

DLS measurements were performed with a DynaPro NanoStar (Wyatt Technology) instrument using a 663 nm laser wavelength. Each sample was introduced in a disposable cuvette (Uvette, Eppendorf) and the measurement was performed by 10 acquisitions of 7 seconds. The data were analyzed with Dynamics 7.1.9 software (Wyatt Technology) to determine the particle size distribution in the submicronic range.

The results are displayed as Z-average, polydispersity index (PDI), monomer radius, percentage of polydispersity of monomer pic, percentage of intensity and of mass of the monomer.

The polydispersity is considered to be low when the PDI is less than 0.1 and high when the sample is multimodal.

The level of homogeneity is considered to be high when the percentage of polydispersity of the monomer is less than 15%. When the level of homogeneity is low (percentage of polydispersity greater than 30%), the particle population can be considered to contain significantly different sizes, or to be polydisperse.

The aggregation is considered to be low when the mass percentage of monomer is more than 99.8% and high when it is less than 99.6%. The aggregation is considered to be low when the intensity percentage of monomer is more than 90.0% and high when it is less than 80.0%.

### Flow Cell Microscopy (FCM)

Flow cell microscopy measurements were performed with a Flowcell FC200-IPAC (Occhio) instrument. Each analysis is performed with 200 μL of sample introduced in a disposable cone. The data were analysed with Callisto software (Occhio) to determine the particle size distribution in the subvisible range.

The results are displayed as the concentration of particles (particles/mL) >2 μm, >10 μm and >25 μm.

The aggregation is considered to be low when the particles >2 μm are less than 2000 per mL, the particles >10 μm are less than 200 per mL and the particles >25 μm are less than 20 per mL. The aggregation is considered to be high when the particles >2 μm are more than 10000 per mL, the particles >10 μm are more than 500 per mL and the particles >25 μm are more than 100 per mL

### Size Exclusion Chromatography (SEC)

SEC measurements were performed with the ultra-high-performance chromatography chain Agilent 1200. The results are displayed as the percentage of high molecular weight (HMW).

For mAb1: The measurements were performed at 24° C. at a flow rate of 0.3 mL/min of mobile phase composed of phosphate buffer 0.1M, NaClO₄ 0.3 M at pH 6.2 and acetonitrile. The samples were stored in vials, and 10 μL were injected in a series of columns composed of guard column Prosec 300S 50×7.5 mm (Agilent) and two columns Prosec 300S 300×7.5 mm (Agilent). The detection was performed at 280 nm. The results were processed with Empower software.

For mAb3: SEC measurements were performed with the ultra-high-performance chromatography chain Agilent 1200. The measurements were performed at 30° C. at a flow rate of 0.3 mL/min of mobile phase composed of 50 mM sodium phosphate, 300 mM sodium perchlorate at pH 7. The samples were stored in vials, and 1 μL were injected in an Aquity UPLC BEH200 SEC 200 Å, 1.7 μm, 4.6 mm×300 mm column (Waters). The detection was performed at 280 nm. The results were processed with Empower software

### Visual Inspection

Visual inspections were performed on samples placed in glass vials and illuminated with an MLC-150 cold light source (Motic) on a black background.

### Example 2: Effect of Buffer and pH on IgG1 Stability

This example shows that acetate and histidine buffer at acidic pH promote the stability of IgG1 antibodies against aggregation upon nebulization, as compared to citrate buffer.

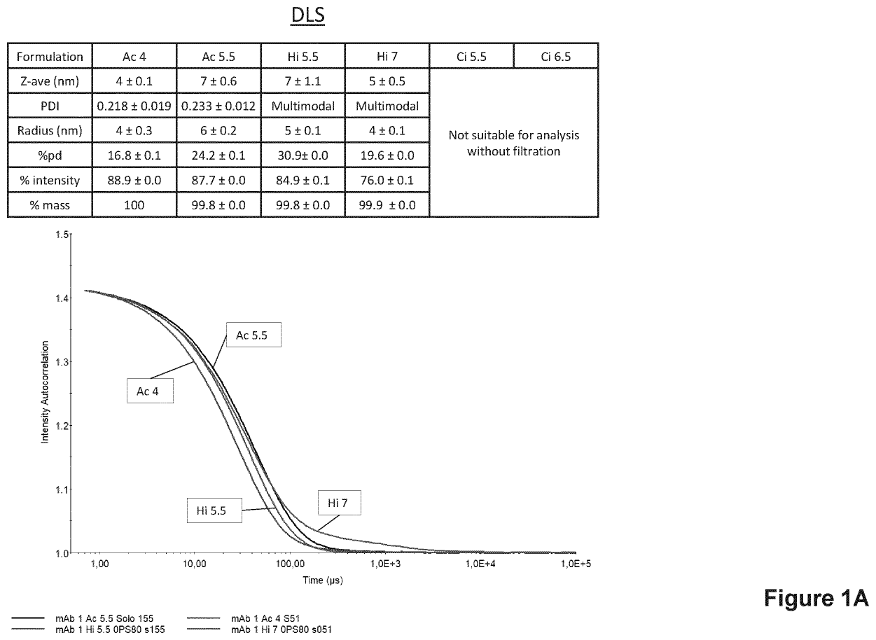

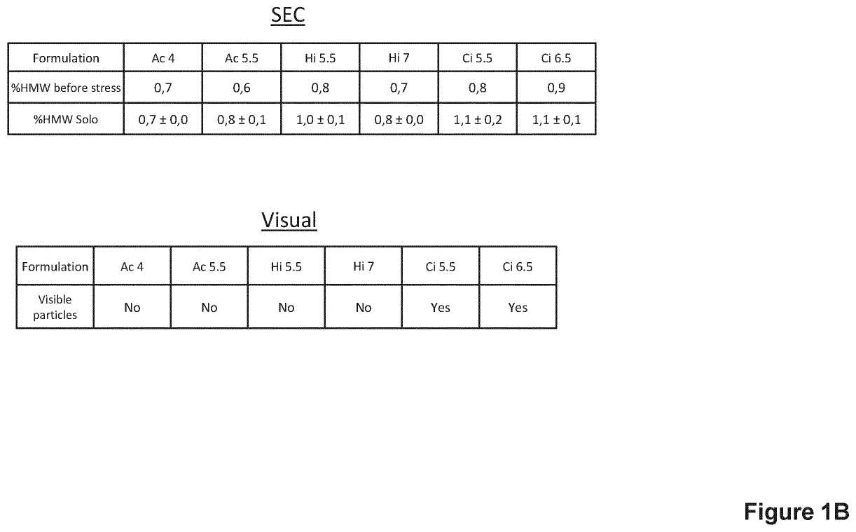

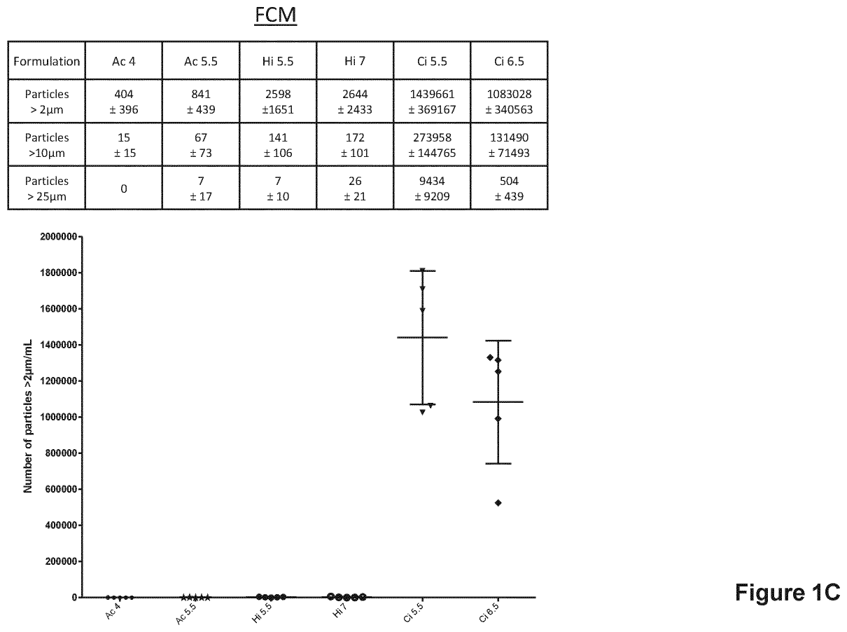

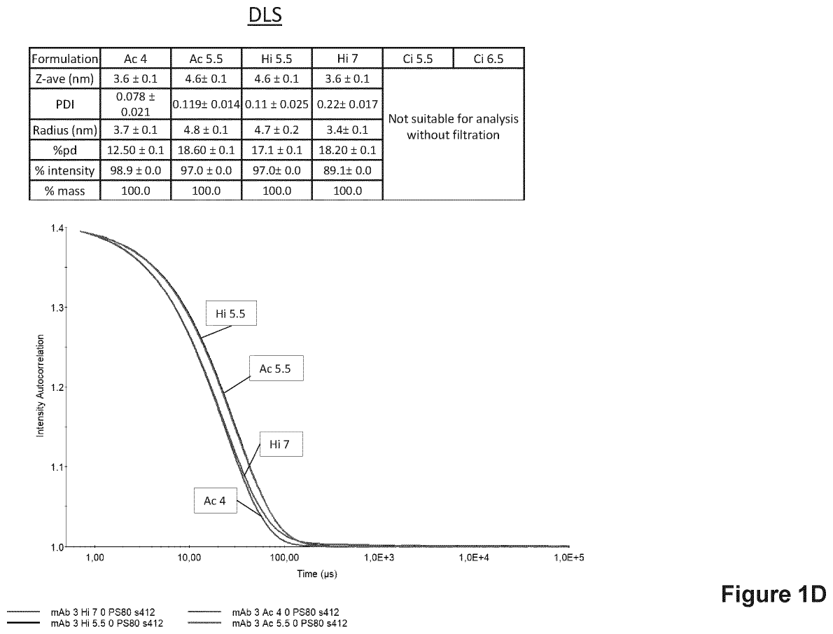

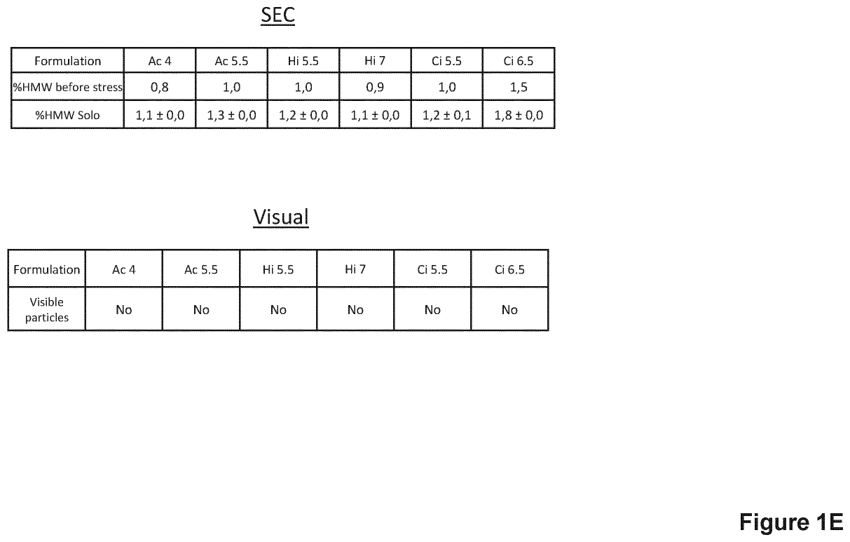

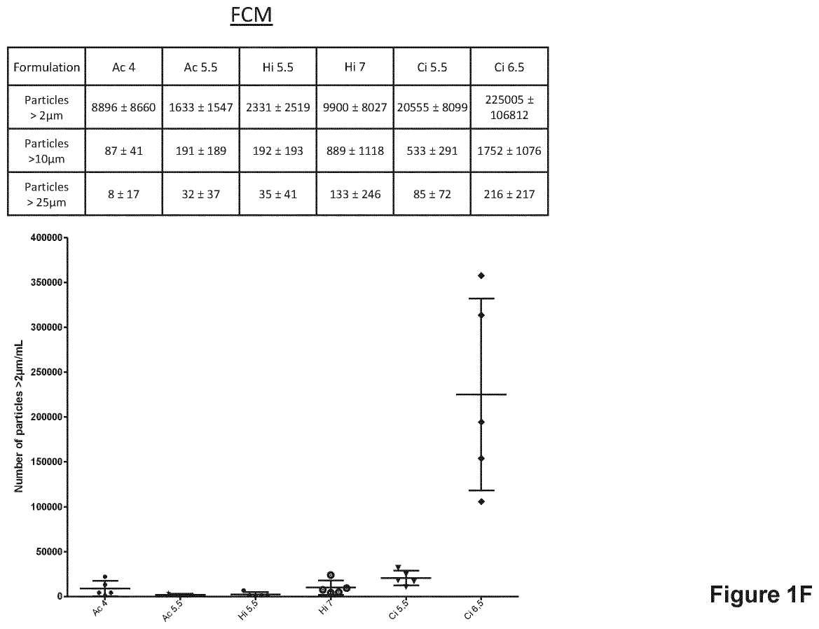

Two IgG1 immunoglobulins (mAb1 and mAb3) formulated in different buffers at different pH without PS80 were nebulized and their stability was characterized after nebulization. mAb1 and mAb3 were formulated at a concentration of 10 mg/mL using six different buffer/pH systems: acetate pH 4.0, acetate pH 5.5, histidine pH 5.5, histidine pH 7.0, citrate pH 5.5 and citrate pH 6.5. Buffer concentrations were 10 mM for each system. Nebulization was performed with a Solo (Aerogen) vibrating mesh nebulizer on 2 mL of IgG1 formulated in the different buffers. The degree of aggregation was measured using dynamic light scattering (DLS), flow cell microscopy (FCM), SEC and visual inspection. The results are summarized in FIGS. 1A-C (mAb1) and FIGS. 1D-F (mAb3).

The visual inspection indicated the presence of visible particles in the citrate formulations of mAb1. The DLS results showed low aggregation into submicron particles in acetate and histidine buffer particularly in acidic pH, whereas citrate formulations were not suitable for analysis without filtration (likely due to high particle content in the samples). The FCM results confirmed high aggregation in citrate buffer.

### Example 3: Effect of Polysorbate 80 (PS80) on IgG1 Stability

This example shows that high amounts of PS80 (1000 ppm) help to limit aggregation upon nebulization of IgG1 in citrate buffer, but not completely as submicron particles of mAb1 are beyond the level of quantification by DLS.

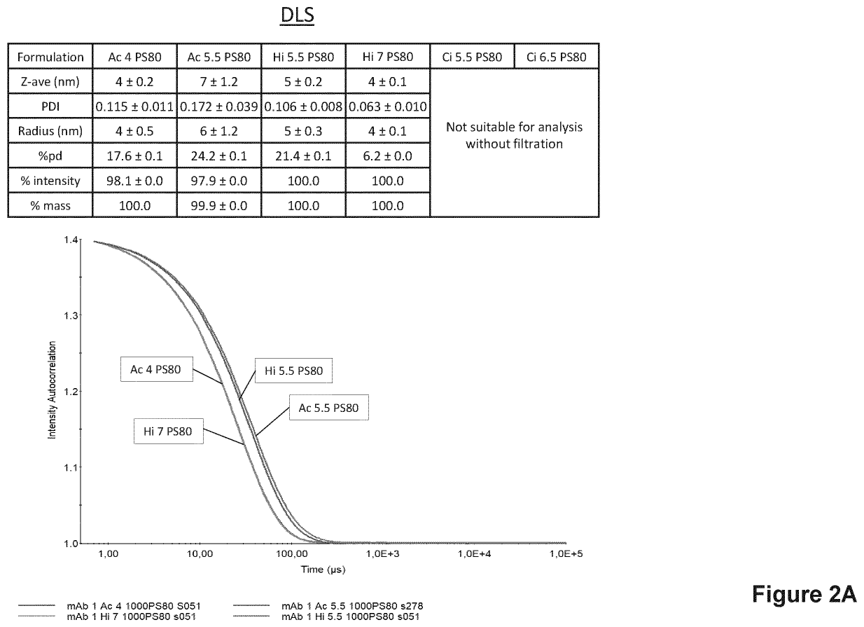

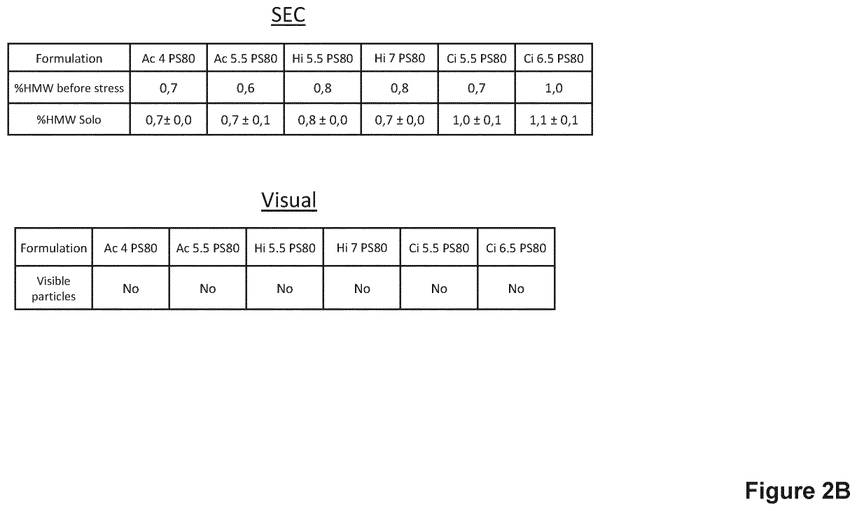

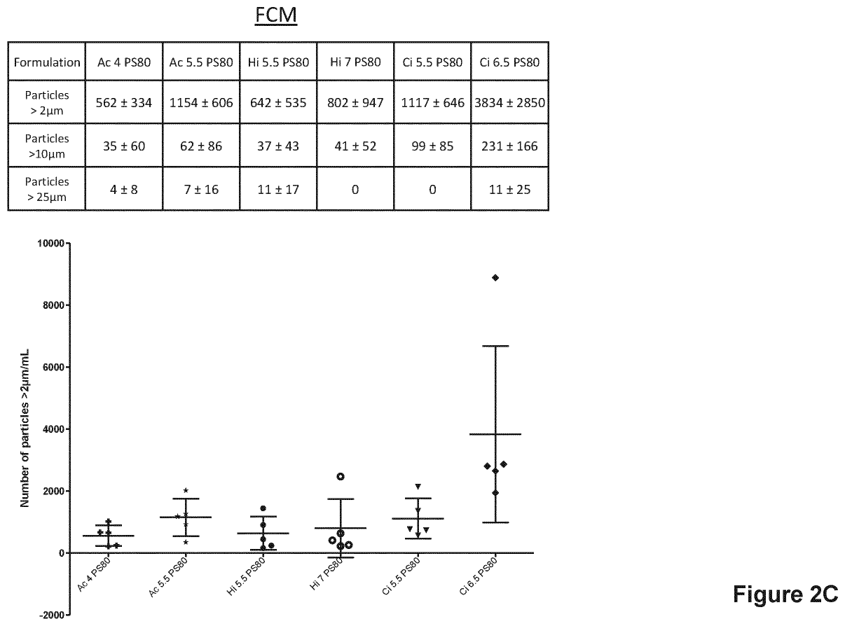

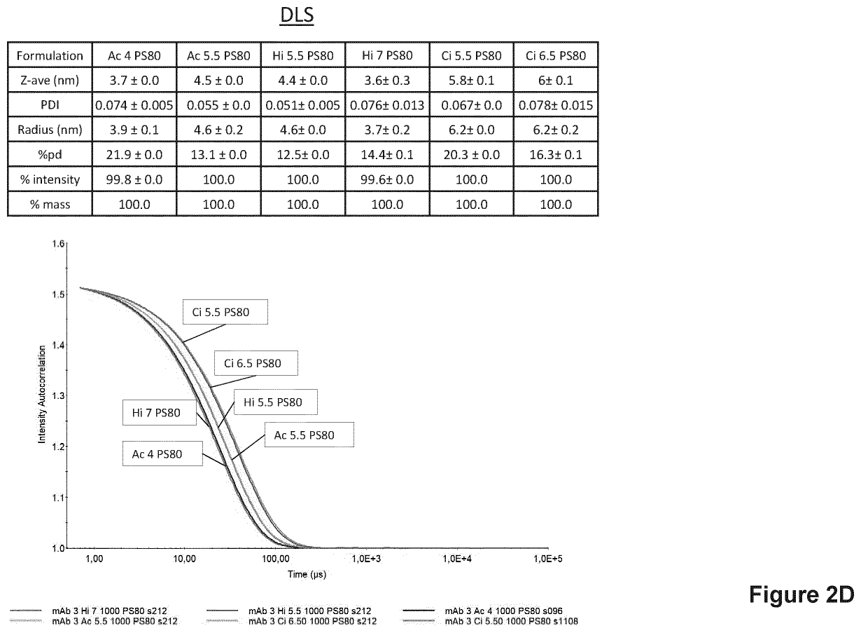

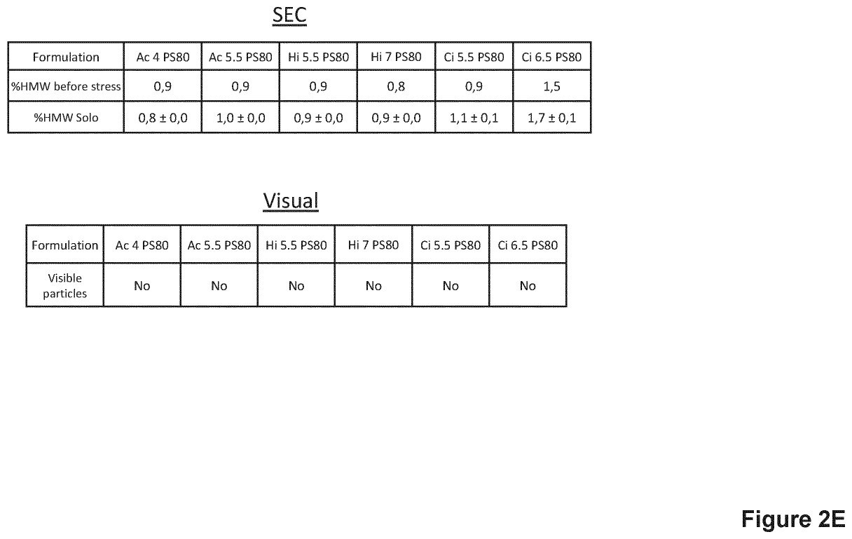

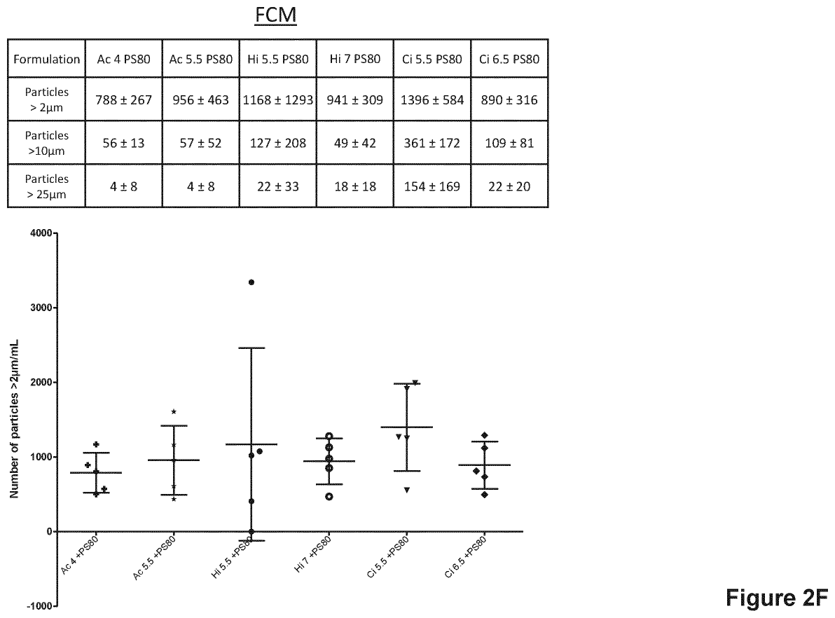

Two immunoglobulins (mAb1 and mAb3) were formulated in different buffers at different pH and in presence of polysorbate 80 (PS80). Acetate, histidine and citrate buffers were employed, and PS80 was used as surfactant. The two IgGs were prepared at a concentration of 10 mg/mL using six different buffer/pH systems: acetate pH 4.0, acetate pH 5.5, histidine pH 5.5, histidine pH 7.0, citrate pH 5.5 and citrate pH 6.5. Buffer concentrations of 10 mM were used. PS80 was added to obtain a concentration of 1000 ppm in to formulations. Nebulization was performed with a Solo (Aerogen) vibrating mesh nebulizer on 2 mL of IgG1 formulated in the different buffers. The degree of aggregation was measured using dynamic light scattering (DLS), flow cell microscopy (FCM), SEC and visual inspection. The results are summarized in FIGS. 2A-C (mAb1) and FIGS. 2D-F (mAb3).

The visual inspection indicated the absence of visible particles in all formulations with PS80. The DLS results showed that citrate formulations were not suitable for analysis without filtration probably due to high particle content/large particles in the samples. The FCM results demonstrated a lower level of aggregation as compared to the same formulations without PS80 tested in Example 2.

### Example 4: Nebulization with Two Different Mesh Vibrating Nebulizers

In this example the stabilizing properties of acetate and histidine buffers are illustrated with two different vibrating mesh nebulizers, namely the Solo nebulizer (see previous examples) and the eFlow nebulizer from PARI. The stabilizing effect on IgG1 antibodies in acetate and histidine buffers (as compared to citrate buffer) were also observed with eFlow system.

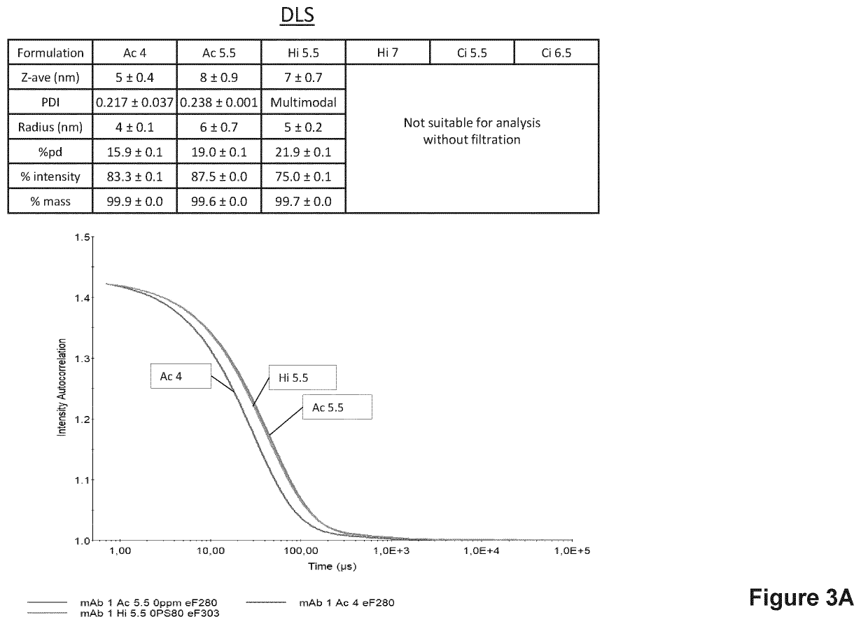

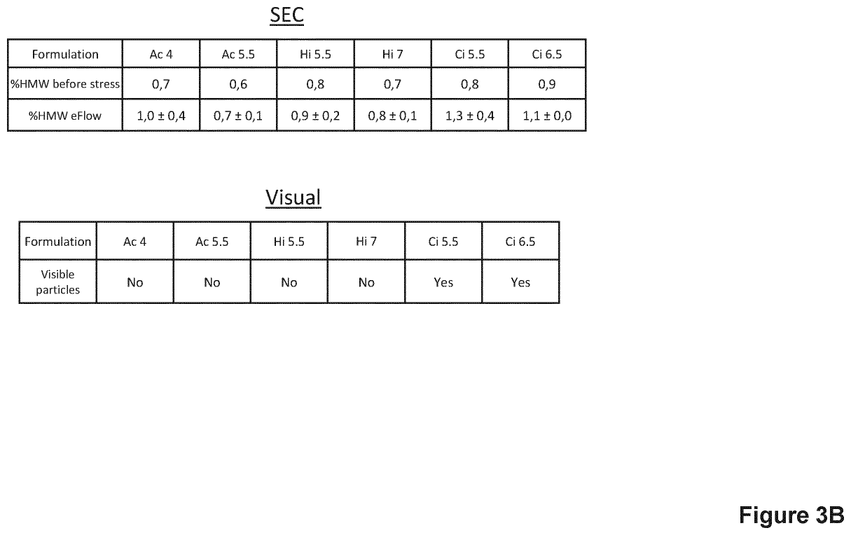

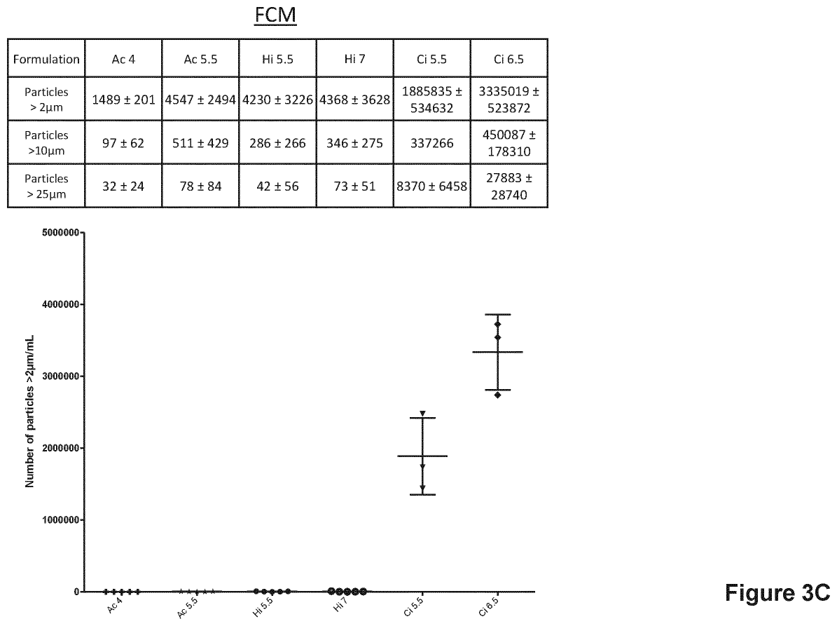

Acetate, histidine and citrate buffers were employed. The two IgG1s were prepared at a concentration of 10 mg/mL using six different buffer/pH systems: acetate pH 4.0, acetate pH 5.5, histidine pH 5.5, histidine pH 7.0, citrate pH 5.5 and citrate pH 6.5. Nebulization stress was applied with a customized eFlow (PARI) vibrating mesh nebulizer to 2 mL of IgG1 formulated in the different buffers. The degree of aggregation was measured using dynamic light scattering (DLS), flow cell microscopy (FCM), SEC and visual inspection. The results are summarized in FIGS. 3A-C (mAb1).

### Example 5: Stability of Different IgG Isotypes (IgG1 and IgG4) in Acetate Buffer pH 5.5

This example illustrates stabilizing effects of acetate buffer at acidic pH on several IgG1 antibodies. The stabilizing properties of these buffer are less noticeable with an IgG4 antibody.

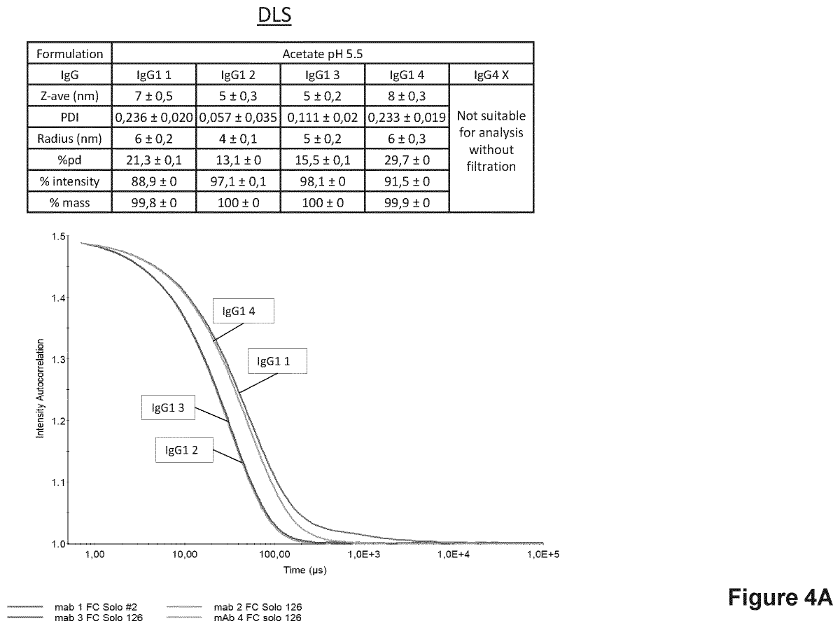

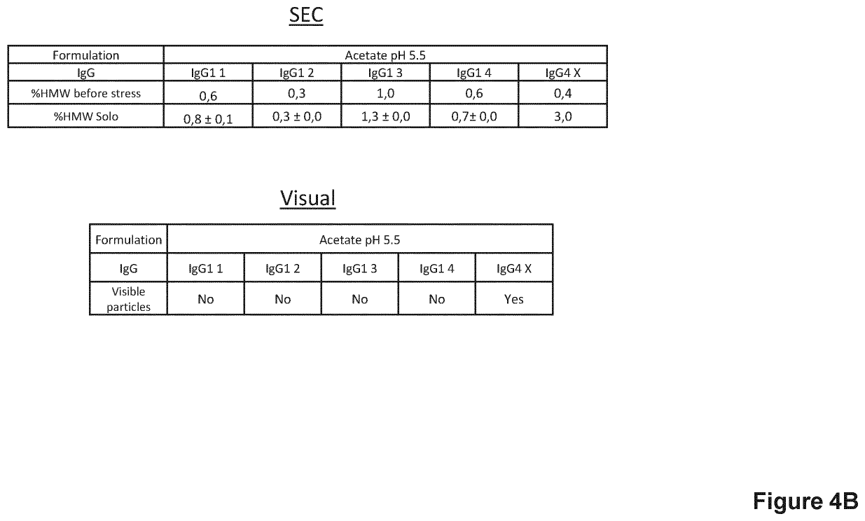

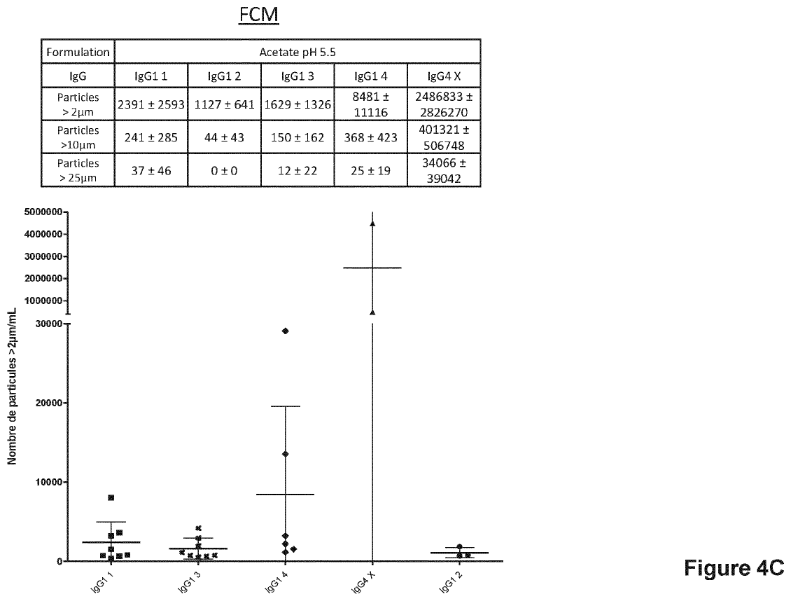

Nebulization stress was applied to four IgG1s (mAb1, mAb2, mAb3 and mAb4) and one IgG4 (mAbX) at a concentration of 10 mg/mL in the same formulation (acetate buffer at pH 5.5). the nebulization was applied with a Solo (Aerogen) vibrating mesh nebulizer on 2 mL. The degree of aggregation was measured using dynamic light scattering (DLS), flow cell microscopy (FCM), SEC and visual inspection. The results are summarized in FIGS. 4A-C.

The visual inspection indicated the presence of visible particles in the IgG4 X sample. The DLS results showed that the IgG4 X sample was not suitable for analysis without filtration probably due to the particles in the samples. FCM counting confirmed a high aggregation in the IgG4 X sample and a low aggregation in the four IgG1 samples.

### Example 6: Stabilizing Effect of Histidine

This example illustrates the stabilizing properties of histidine buffer in a formulation containing additional excipients (a polyol and a non-ionic surfactant) allowing manufacturing and long-term storage of the drug product before use for nebulization.

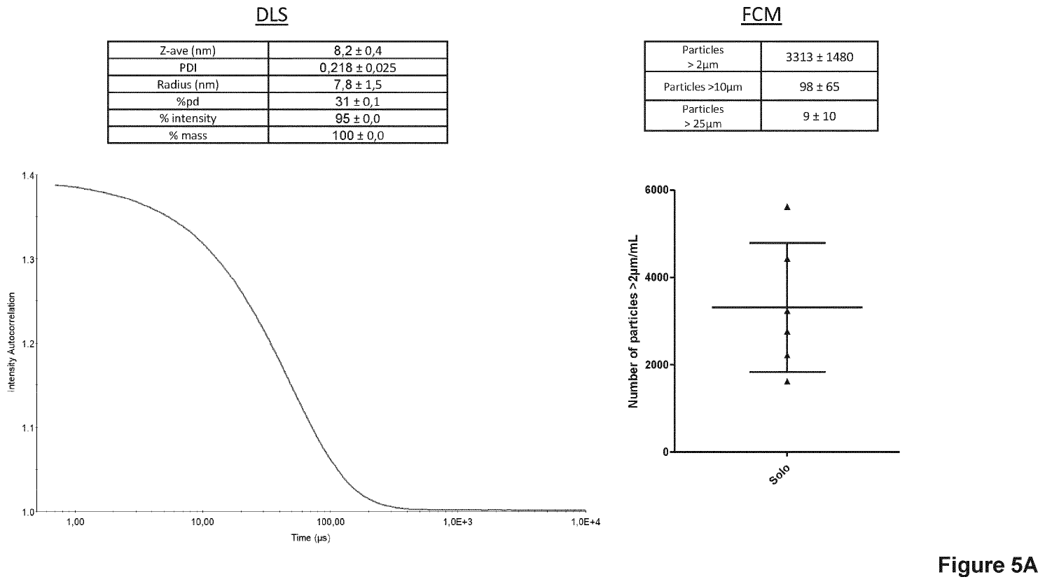

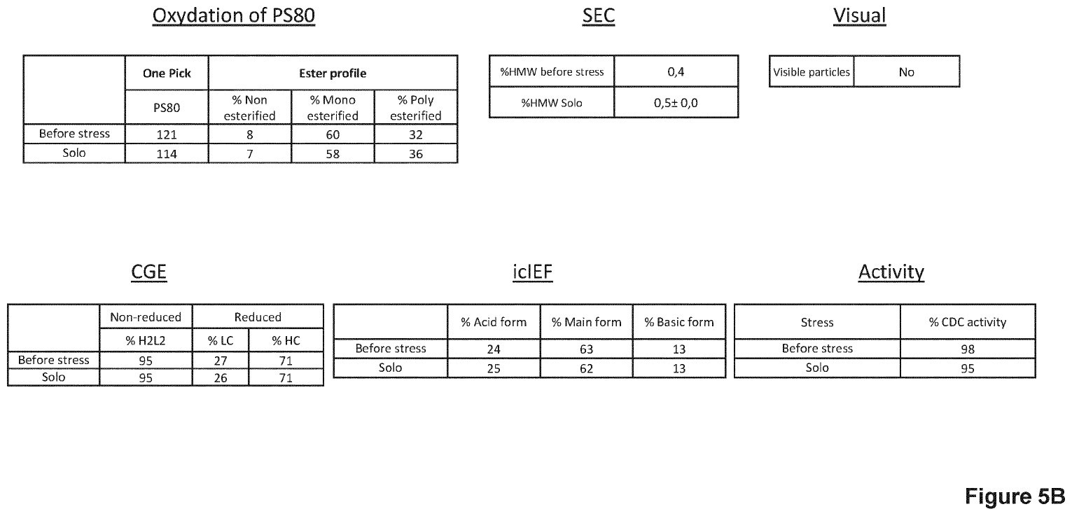

The IgG1 antibody (mAb1) was formulated at 20 g/L in 20 mM histidine buffer at pH 6.0 with a non-ionic surfactant and a polyol as additional excipients, a combination which provides long-term storage stability to the drug product solution. Nebulization stress was applied with a Solo (Aerogen) vibrating mesh nebulizer to 2 mL of the mAb1 formulation. The aerosol droplet size was determined by laser diffraction measurements. The VMD obtained after nebulization was 4.4 m, and most of the droplets had a diameter below 5 μm. About 30% of the droplets had a diameter in the range of 0.5 μm to 3 μm. The degree of aggregation was characterized by dynamic light scattering (DLS), flow cell microscopy (FCM), SEC and visual inspection. Additional analytical methods were performed. The formulation can be stored at 2-8° C. over 24 months. The results are summarized in FIGS. 5A and 5B.

### Example 7: Effect of PS80 Concentration on IgG1 in Citrate Buffer

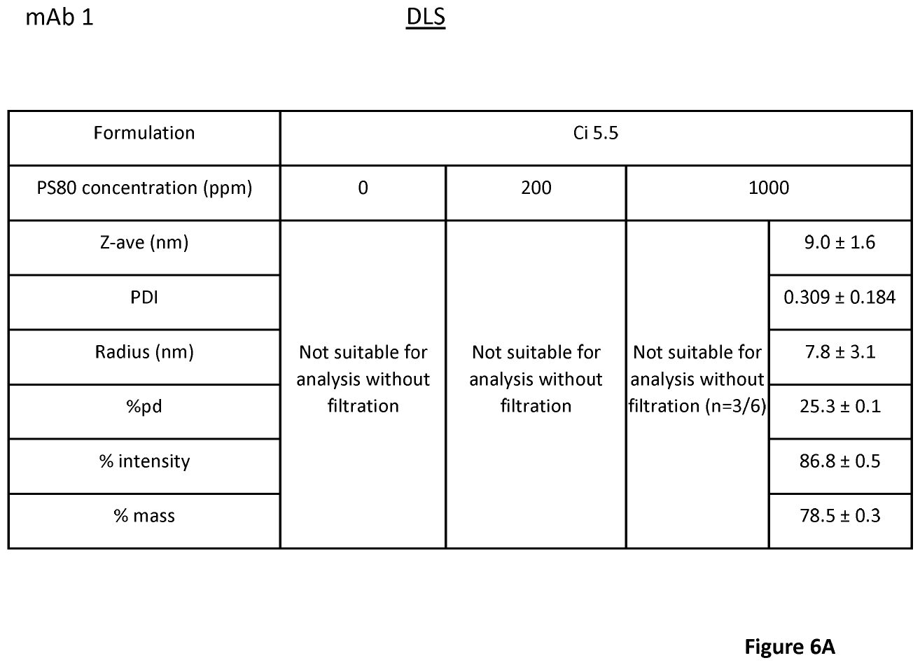

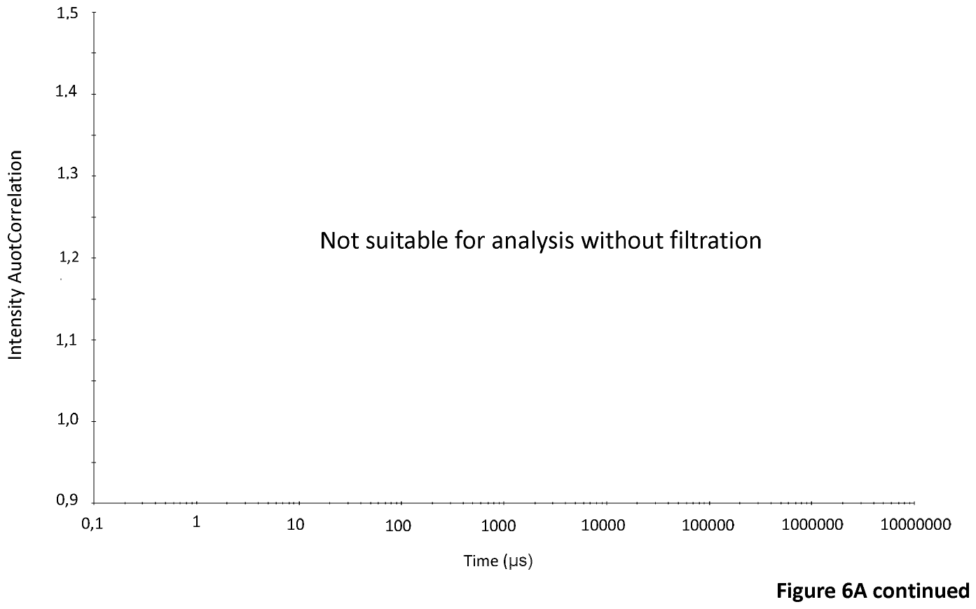

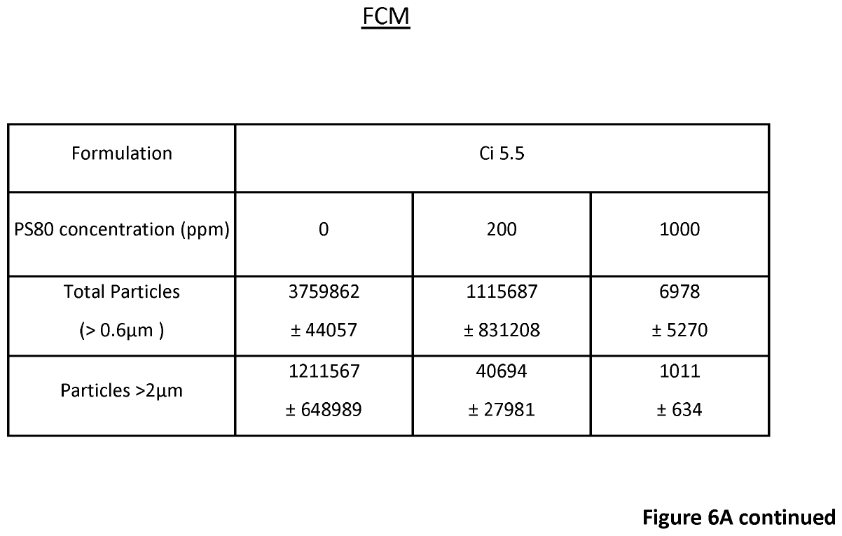

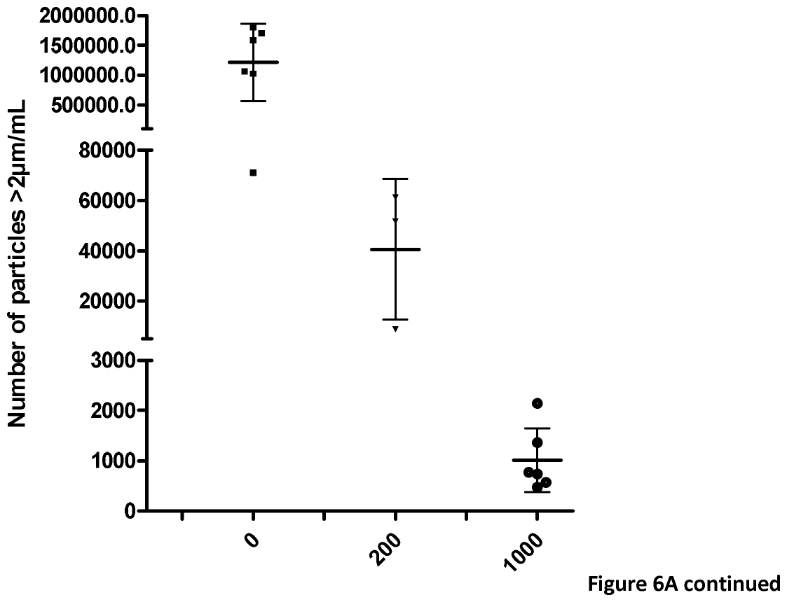

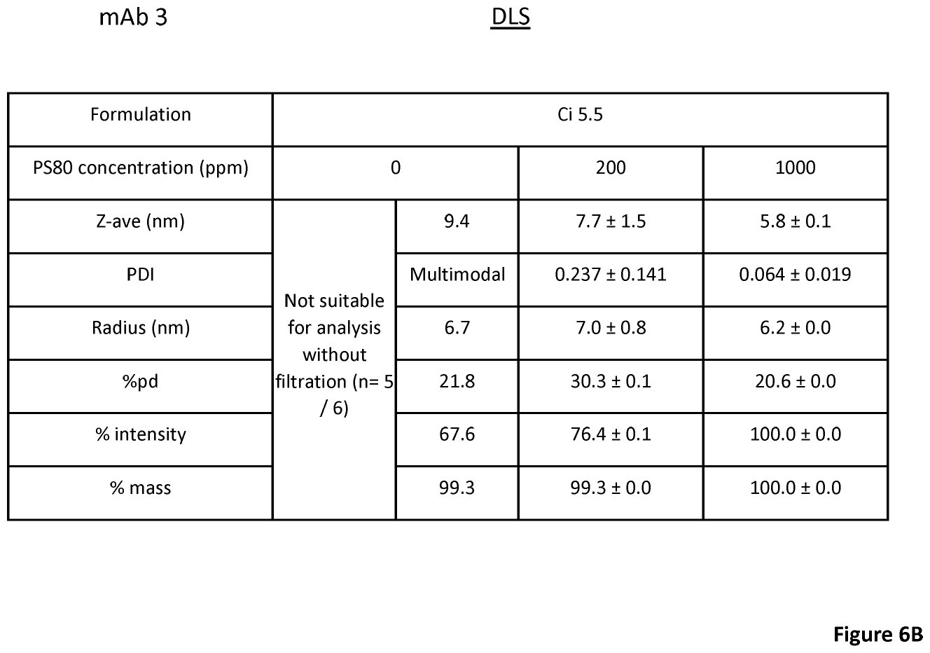

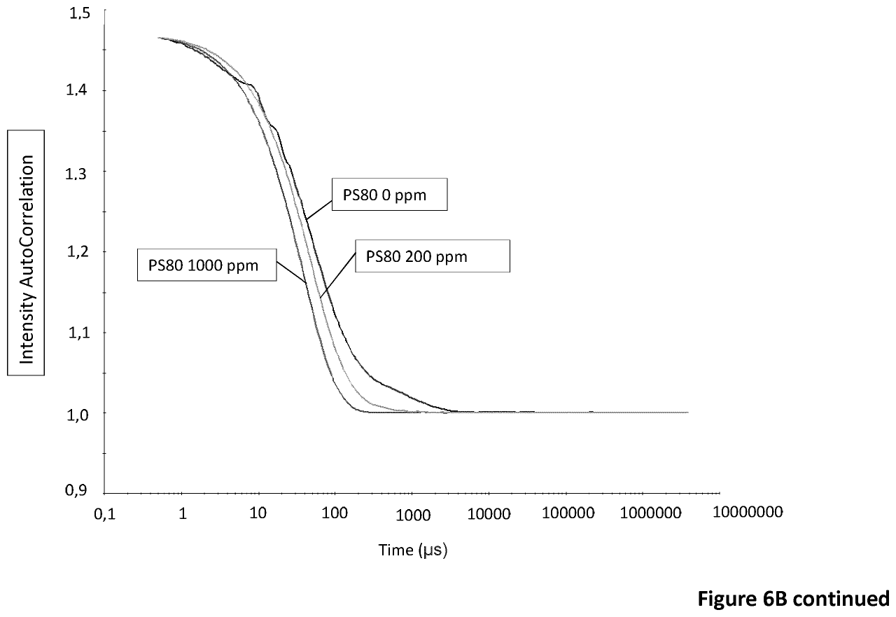

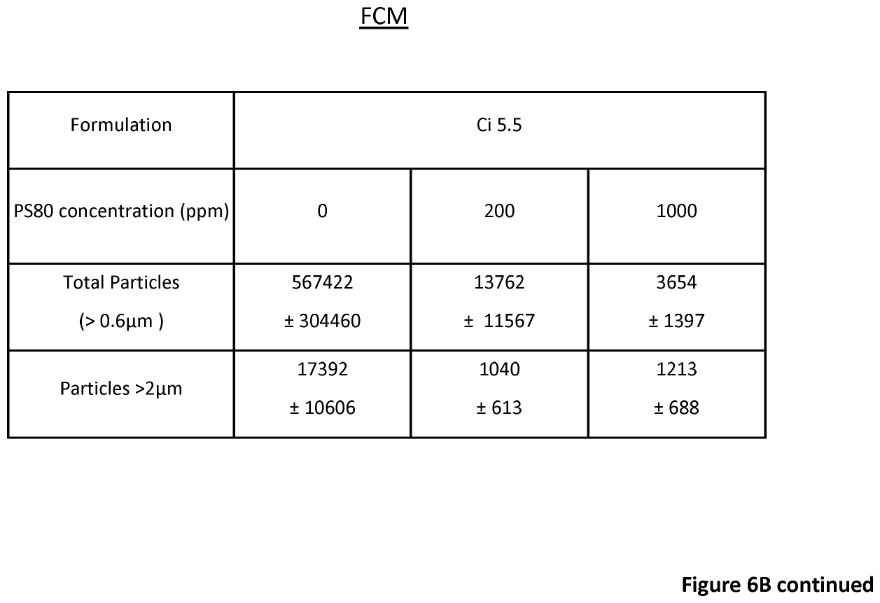

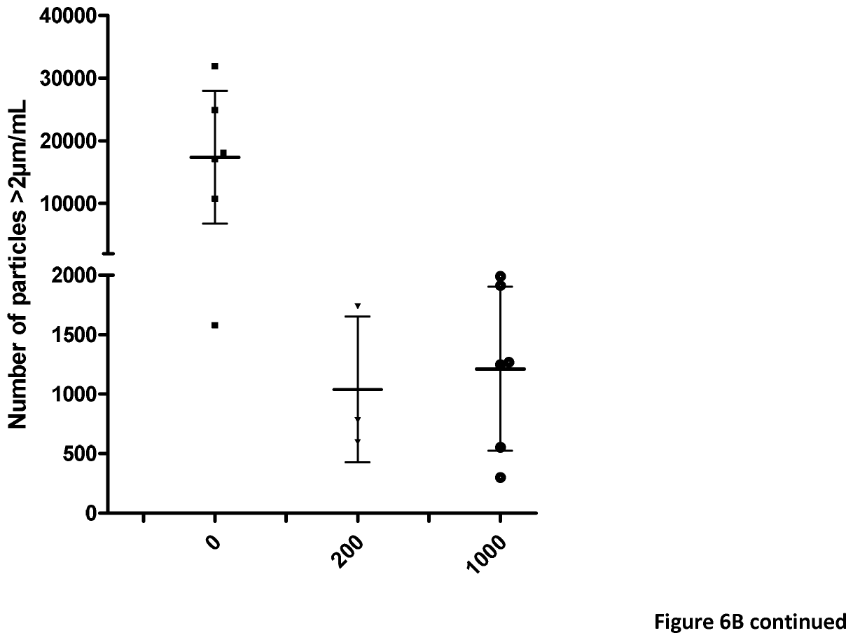

The effect of PS80 concentration on the stability of IgG1 nebulized in citrate was evaluated. In this regard, nebulization stress was applied to two different IgG1 (mAb 1 and mAb 3) formulated in citrate pH 5.5 with different concentrations of PS80. 0, 200 and 1000 ppm of PS80 were employed. The two IgG1 were prepared at a concentration of 10 mg/mL in citrate buffer and PS80 was added at the target concentrations. Nebulization stress was applied with a vibrating mesh nebulizer on 2 mL of IgG1 formulated in citrate with different concentrations of PS80. The degree of aggregation was measured using a Dynamic Light Scattering (DLS), Flow Microscopy (FCM; FIG. 6). FIG. 6 shows that IgG1 in citrate buffer presented a high level of aggregation with 0 ppm of PS80. High concentrations (1000 ppm) of PS80 were required to stabilize IgG1 in citrate. The DLS results show that citrate formulations are not suitable for analysis without filtration probably due to the particles in the samples. The FCM results reveal a high concentration of particles with 0 ppm of PS80. The number of particles per mL decreased when the concentration of PS80 increased. 200 ppm of PS80 was not sufficient to stabilize mAb 1. The DLS results show that citrate formulations are not suitable for analysis without filtration probably due to particles in the samples at 0 ppm of PS80 in most of the case (n=5/6). At 200 ppm of PS80, aggregation is observed. At 1000 ppm of PS80, aggregation is low. The FCM results reveal a high concentration of particles with 0 ppm of PS80. Addition of PS80 at 200 and 1000 ppm decreased the concentration of particles.

### Example 8: Effect of PS80 Concentration on IgG1 in Histidine Buffer

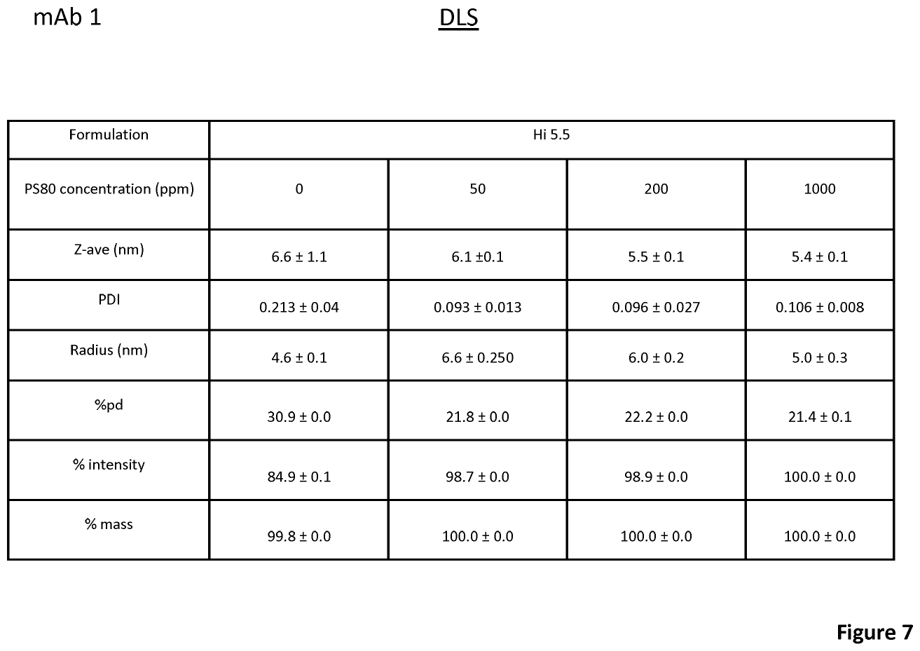

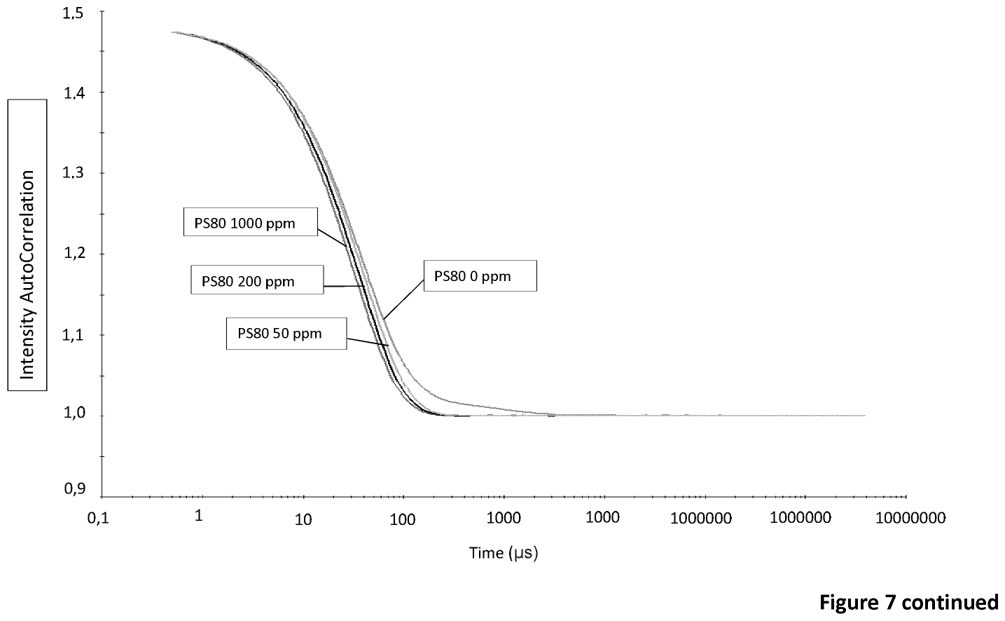

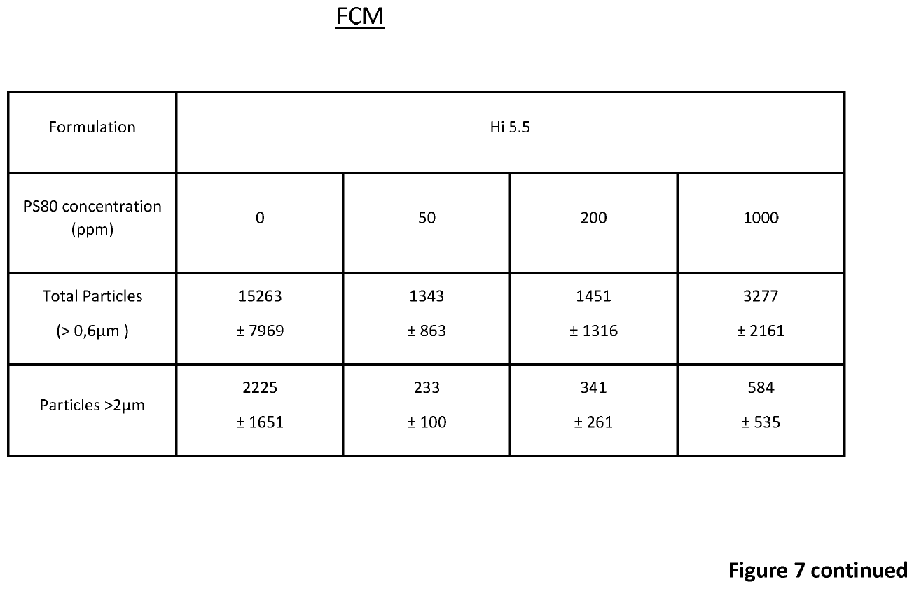

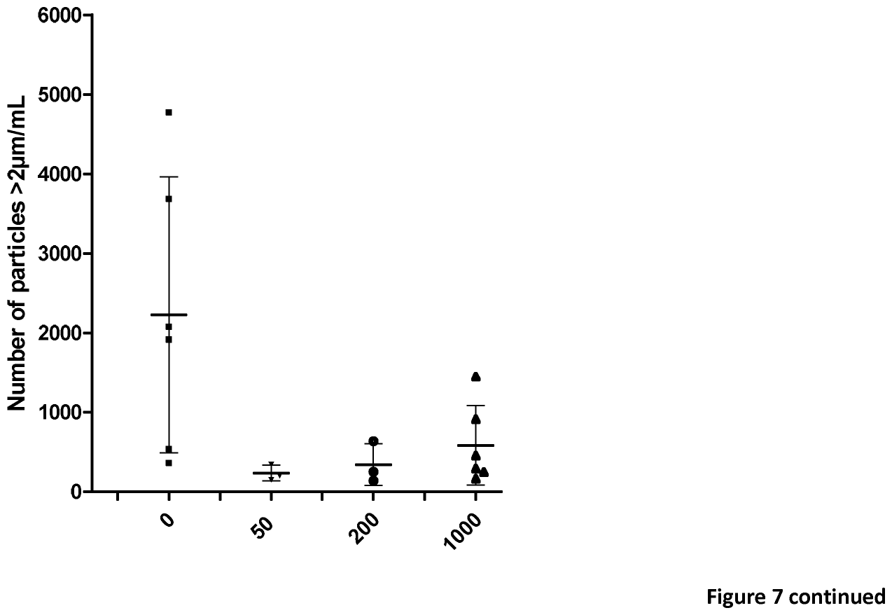

The effect of PS80 concentration on the stability of IgG1 nebulized in histidine was evaluated. In this regard, nebulization stress was applied to one IgG1 (mAb 1) formulated in histidine pH 5.5 with different concentrations of PS80. 0, 50, 200 and 1000 ppm of PS80 were employed. The IgG1 was prepared at a concentration of 10 mg/mL in histidine buffer and PS80 was added at the target concentrations. Nebulization stress was applied with a vibrating mesh nebulizer on 2 mL of IgG1 formulated in histidine with different concentrations of PS80. The degree of aggregation was measured using Dynamic Light Scattering (DLS) and Flow Microscopy (FCM; FIG. 7). FIG. 7 shows that IgG1 in histidine buffer presented a moderate level of aggregation with 0 ppm of PS80. Low concentrations of PS80 (50 ppm) appears to be sufficient to stabilize IgG1 in histidine. The DLS results show a moderate aggregation at 0 ppm of PS80. At 50, 200 and 100 ppm of PS80, the level of aggregation is low and similar among the different concentrations. The FCM results reveal a moderate concentration of particles with 0 ppm of PS80. Addition of PS80 at 50, 200 and 1000 ppm allowed to reduce the concentration of particles in a comparable way.

1. An aerosol comprising droplets comprising a liquid formulation,
wherein the liquid formulation comprises
(i) an antibody or antibody derivative,
(ii) a buffering agent selected from the group consisting of acetate, histidine and combinations thereof, and
(iii) an aqueous medium; and
wherein the liquid formulation has a pH which is equal to or lower than about 5.5.

2. The aerosol of claim 1, wherein the liquid formulation does not comprise citrate.

3. The aerosol of claim 1, wherein the buffering agent is acetate, and the liquid formulation has a pH which is lower than about 5.0 or lower than about 4.5.

4. The aerosol of claim 1, wherein the liquid formulation further comprises a surfactant.

5. The aerosol of claim 4, wherein the concentration of the surfactant in the liquid formulation is equal to or lower than about 0.1% (w/v) or equal to or lower than about 0.05% (w/v).

6. The aerosol of claim 4, wherein the surfactant is selected from the group consisting of polysorbates, poloxamers, polyoxyethylenealkylethers, alkylphenolpolyoxyethylene and sodium dodecyl sulfate.

7. The aerosol of claim 1, wherein the liquid formulation does not comprise any surfactant.

8. The aerosol of claim 1, wherein the antibody or antibody derivative is an IgG1 antibody or antibody derivative.

9. The aerosol of claim 1, wherein the droplets have an average diameter in the range of from about 0.5 μm to about 5 μm, from about 0.5 μm to about 4.5 μm, from about 0.5 μm to about 4 μm, from about 0.5 μm to about 3.5 μm or from about 0.5 μm to about 3 μm.

10. A method of preparing an aerosol comprising droplets comprising a liquid formulation, said method comprising the steps:
(i) providing a liquid formulation as defined in claim 1,
(ii) nebulizing the liquid formulation provided in step (i) by means of a nebulizer, thereby preparing the aerosol.

11. The method of claim 10, wherein, optionally, said method further comprises, between steps (i) and (ii), the steps of:
(ia) lyophilizing the liquid formulation provided in step (i), thereby providing a lyophilized powder, and
(ib) reconstituting the liquid formulation provided in step (i) by adding an appropriate amount of an aqueous medium to the lyophilized powder provided in step (ia).

12. An aerosol comprising droplets comprising a liquid formulation, wherein the aerosol is obtainable by the method of claim 10.

13. The aerosol of claim 1 for use in a method of delivering an antibody or antibody derivative to the lungs of a subject, wherein the aerosol is administered to the subject by inhalation or the liquid formulation is administered to the subject by inhalation via a nebulizer.

14. The aerosol of claim 1 for use in a method of treating or preventing a disease in a subject, wherein the aerosol is administered to the subject by inhalation or the liquid formulation is administered to the subject by inhalation via a nebulizer.

15. A nebulizer comprising a liquid formulation as defined in claim 1.

16. A kit comprising
(i) a container comprising the liquid formulation as defined in claim 1 or a powder obtainable by lyophilization of the liquid formulation, and
(ii) a nebulizer.

17. A method for preparing an aerosol by nebulization, said method comprising the steps:
(i) providing a liquid formulation as defined in claim 1,
(ii) nebulizing the liquid formulation provided in step (i) by means of a nebulizer, thereby preparing the aerosol.

18. A method for increasing the stability of an antibody or antibody derivative upon nebulization of a liquid formulation comprising the antibody or antibody derivative by means of a nebulizer, the method comprising:
adding a buffering agent to the liquid formulation prior to nebulization, wherein the buffering agent is selected from the group consisting of acetate, histidine and combinations thereof.
